(19) United States
(12) Patent Application Publication
(10) Pub. No.: US 2005/0142585 A1
(43) Pub. Date: Jun. 30, 2005

(54) Determination of phenotype of cancer and of precancerous tissue

(75) Inventor: Gerold Bepler, Tampa, FL (US)

Correspondence Address: JONES DAY, 222 EAST 41ST ST, NEW YORK, NY, 10017, US

(73) Assignee: University of South Florida

(21) Appl. No.: 10/957,844

(22) Filed: Oct. 4, 2004

Related U.S. Application Data
(60) Provisional application No. 60/508,055, filed on Oct. 2, 2003.

(51) Int. Cl. C12Q 1/68

(52) U.S. Cl. 435/6

(57) ABSTRACT

The present invention relates to methods for determining and/or predicting the phenotype of a cancer or precancerous tissue. In certain embodiments, the methods described herein relate to predicting of survival of a subject with a cancer or a precancerous tissue, predicting response to therapy of a subject with a cancer or precancerous tissue, predicting metastasis of a cancer in a subject, predicting recurrence of cancer in a subject, or predicting the progression of a precancerous tissue to cancer. The present invention further relates to kits for determining and/or predicting the phenotype of a cancer or a precancerous tissue.

This application claims benefit under 35 U.S.C. § 119(e) of U.S. Provisional Application Ser. No. 60/508,055, filed Oct. 2, 2003, which is incorporated herein by reference in its entirety.

This invention was made with government support under grant number DAMD 17-02-2-0051 awarded by the Department of Defense. The United States Government has certain rights in the invention.

### FIELD OF THE INVENTION

The present invention relates to methods for determining and/or predicting the phenotype of a cancer. In certain embodiments, the methods described herein relate to predicting survival of a subject with a cancer, predicting response to therapy of a cancer in a subject, predicting metastasis of a cancer in a subject, and/or predicting recurrence of cancer in a subject. The present invention further relates to kits for determining and/or predicting the phenotype of a cancer.

### BACKGROUND OF THE INVENTION

It is well established that genome damage is a factor in cancer (Yunis, 1983, Science 221:227-236). Damage to DNA has been linked to causes such as increased chromosome fragility and/or impaired repair of DNA strand breaks during cell cycle progression (Hoeijmakers, 2001, Nature 411:366-374). Other causes of genome damage include gene mutation and altered transcription through mutations or epigenetic modifications in regulatory elements (Vogelstein et al., 1988, N Engl J Med 319:525-532).

Several approaches have been taken to assess the relationship of global genome damage and cancer. Early studies focused on the relative content of DNA in tumor cells as compared to normal cells based on the observation that many tumors exhibited aneuploidy (Barlogie et al., 1982, Cancer Genet Cytogenet 6:17-28; Wolley et al., 1982, Natl Cancer Inst 69:15-22; Auer et al., 1984, Cancer Res 44:394-396; Volm et al. 1985, Cancer 56:1396-1403). Once genes and chromosomal regions or loci were discovered that contained or were thought to contain genes relevant to cancer biology, studies assessing changes in heterozygosity of alleles of one or more of such genes in cancerous versus non-cancerous tissues were undertaken (Cavenee et al., 1983, Nature 305:779-784; Ali et al., 1987, Science 238:185-188; Jen et al., 1994 N Engl J Med 331:2123-221; Fong et al., 1995, Cancer Res 55:220-223; Mitsudomi etal., 1996, Clin Cancer Res 2:1185-1189; and Bepler et al., 2002, J Clin Oncol 20:1353-1360). These studies involved use of markers such as restriction fragment length polymorphisms (RFLPs), minisatellites, mircosatellites, and simple nucleotide repeat polymorphisms to examine loss of polymorphism (i.e. loss of heterozygosity) at specific loci in tumor DNA. Many of these markers introduce bias to analyses of global genome damage in that their locations tend to cluster around telomeres rather than being randomly distributed throughout the genome. Use of loss of heterozygosity in single or multiple loci that contain genes important to tumor biology was examined as a potential marker for tumor phenotype in order to predict tumor behavior. For example, Bepler et al., (2002, J Clin Oncol 20:1353-1360) found that loss of heterozygosity at chromosome segment 11 p15.5, known to contain genes involved in cancer biology, is correlated with the metastatic spread of lung cancer and poor survival. Attempts to assess global genome damage examined limited numbers of loci with an emphasis on loci thought to be involved in cancer biology. Vogelstein et al. (1998, Science 244:207-211) examined a locus on each arm of each human chromosome in colorectal carcinoma samples and found a median loss of heterozygosity of 20%, with patients having greater than 20% exhibiting shorter survival. Vogelstein et al. (U.S. Pat. No. 5,580,729, dated Dec. 3, 1996) uses RFLP analysis, assessing the change in size of restriction enzyme digestion fragments, to assess fractional allele loss, particularly in colorectal cancer.

There exists a need for a high resolution, highly sensitive method for assessment of global genome damage that can be used to determine and/or predict the impact of such damage on the in vivo behavior of cancers.

### SUMMARY OF INVENTION

The present invention provides methods for determining and/or predicting the phenotype of a cancer. The phenotype can be, for example, predicting survival of a subject with a cancer, predicting response to therapy of a subject with a cancer, predicting metastasis of a cancer in a subject, or predicting recurrence of cancer in a subject. The present invention further relates to kits for determining and/or predicting the phenotype of a cancer.

The invention provides a method for assessing global genome damage through determining the extent of loss of heterozygosity among single nucleotide polymorphisms (hereafter “SNPs”) that are randomly distributed throughout the genome (i.e., not biased towards specific chromosomal loci, although biases such as avoidance of repetitive DNA can be used in the selection of the SNPs) and whose association with cancer was not predetermined. The SNPs are thus non-specific, independent of particular genes or loci. The present invention has yielded the unexpected discovery that global genome damage is lower in cancers than what would have been predicted based on extrapolation of measurements of loss of heterozygosity found in the prior art, which employed techniques that were less comprehensive in coverage of the genome and that were biased toward examination of certain chromosomal loci (known or suspected to be associated with cancer). Furthermore, it has been determined through use of the present invention that the damage to genomic DNA in cancer was distributed genome-wide to an extent that one would not have predicted based on the prior art. The accuracy of prediction of the phenotype of a cancer is enhanced using the methods of the invention described herein.

The advantages of the methods of the invention include the more accurate prediction of poor or positive prognosis. These advantages will greatly impact clinical trials for cancer therapies, because potential study patients can be stratified according to prognosis. Trials can then be limited to patients having poor prognosis, in turn making it easier to discern if an experimental therapy is efficacious. It would, therefore, be beneficial to provide specific methods for the prognosis, of cancer and to provide methods that would identify individuals with a predisposition for the onset of cancer and hence are appropriate subjects for preventive therapy.

According to one aspect the invention provides for a method for determining phenotype of a cancer in a subject comprising determining a global genome damage score (hereinafter “GGDS”) for the cancer, wherein said GGDS is a relative measure of (a) number of heterozygous single nucleotide polymorphisms (“SNPs”) in a plurality of heterozygous SNPs, said plurality of heterozygous SNPs consisting of different SNPs wherein heterozygosity occurs in genomic DNA of non-cancerous tissue of said species to which said subject belongs, wherein said number of heterozygous SNPs in said plurality is in excess of 100 SNPs, and (b) the number of SNPs for which heterozygosity is determined to be present, or the number of SNPs for which heterozygosity is determined to be absent, among the number of heterozygous SNPs in said plurality of (a), in a nucleic acid sample of, or derived from, genomic DNA of cancerous tissue of the subject. The GGDS can be compared to one or more threshold values with the GGDS being above (or alternatively below) the threshold value(s) being indicative of the phenotype. In certain embodiments of this method, the number of SNPs in part (b), for which heterozygosity is determined to be present or for which heterozygosity is determined to be absent, is determined by a second method comprising a) contacting under hybridization conditions said nucleic acid sample of, or derived from, genomic DNA of cancerous tissue of the subject independently with each member of a SNP pair, for each heterozygous SNP in said plurality of heterozygous SNPs, each SNP pair being a pair of oligonucleotides differing in sequence at a single nucleotide position that is a site of a single nucleotide polymorphism, and b) detecting any hybridization that occurs.

In certain embodiments, the plurality of heterozygous SNPs used in the methods of the invention to determine the phenotype of a cancer comprises heterozygous SNPs comprising a nucleotide sequence complementary to the genomic DNA sequence of at least 100 different loci in said species. In certain embodiments, the plurality of heterozygous SNPs used in the methods of the invention to determine the phenotype of a cancer comprises at least 100 heterozygous SNPs that are randomly distributed throughout the genome at least every 500 kb. In certain embodiments, the plurality of heterozygous SNPs used in the methods of the invention to determine the phenotype of a cancer comprises at least 100 heterozygous SNPs that are not within the same 500 kb region of said genomic DNA as any other SNPs within said plurality. In certain embodiments, the plurality of heterozygous SNPs comprise at least 500 SNPs that are not within the same 500 kb region of said genomic DNA as any other SNPs within said plurality. In certain embodiments, the number of heterozygous SNPs in said plurality is in excess of 500. In certain embodiments, the number of heterozygous SNPs in said plurality is in excess of 1000.

According to certain aspects of the invention, the plurality of heterozygous SNPs used in the methods of the invention to determine the phenotype of a cancer are not found in regions of genomic DNA that are repetitive. In preferred embodiments, the plurality of heterozygous SNPs comprises at least one SNP on each of the 23 human chromosomes pairs. In other preferred embodiments, the plurality of heterozygous SNPs comprises at least one SNP on each arm of each of the 23 human chromosomes pairs. In certain embodiments, the plurality of heterozygous SNPs comprises SNPs, located in the genome on different chromosomal loci, respectively, and wherein the different chromosomal loci comprise are on each of the chromosomes of said species.

In one embodiment, the non-cancerous tissue used in the methods of the invention is derived from the same tissue type as the cancerous tissue. In another embodiment, the non-cancerous tissue is not the same tissue type as said cancerous tissue. In other embodiments, the non-cancerous tissue is derived from mononuclear blood cells or saliva cells. In yet other embodiments, the non-cancerous tissue is from a plurality of different organisms. In still other embodiments, the non-cancerous tissue is from the subject. In preferred embodiments of the methods of the invention, the subject is human.

In one embodiment, tissue from potentially pre-cancerous lesions is used in the methods of the invention rather than cancerous tissue so that a GGDS predictive of the probability of developing cancer is determined.

In certain embodiments, the number of SNPs in part (b) of the methods of the invention, for which heterozygosity is determined to be present or for which heterozygosity is determined to be absent, is determined by a method that does not comprise detecting a change in size of restriction enzyme-digested nucleic acid fragments. In certain embodiments, the relative measure is the number of said SNPs in part (b) of the methods of the invention described above for which heterozygosity is determined to be absent divided by the number of heterozygous SNPs in said plurality in part (a) of the methods of the invention.

In certain preferred embodiments, the cancer, the phenotype of which is determined by the methods of the invention, is an epithelial cancer. In related embodiments, the epithelial cancer is breast cancer, prostate cancer, lung cancer, or colon cancer. In related embodiments, the lung cancer is non-small cell lung carcinoma. In certain embodiments, the phenotype of a cancer determined by the methods of the invention is predicted response to therapy. In related embodiments, the therapy is chemotherapy or radiation therapy. In other embodiments, the therapy is immunotherapy. In certain embodiments, the phenotype of a cancer determined by the methods of the invention is predicted probability of survival. In certain embodiments, the phenotype of a cancer determined by the methods of the invention is predicted probability of metastasis within a given time period. In certain embodiments, the phenotype of a cancer determined by the methods of the invention is the predicted probability of tumor recurrence.

In one embodiment, the second method described above further comprises prior to said contacting step the step of producing said nucleic acid sample by a third method comprising amplifying genomic DNA of cancerous tissue of the subject.

The invention also provides a kit comprising (a) nucleic acid probes comprising SNP hybridization probes, said SNP hybridization probes comprising nucleotide sequences complementary to a plurality of SNPs, respectively, said SNPs consisting of at least 100 different SNPs wherein heterozygosity occurs in genomic DNA of non-cancerous tissue of the same species; and (b) a computer program product for use in conjunction with a computer system, the computer program product comprising a computer readable storage medium and a computer program mechanism embedded therein, the computer program mechanism comprising instructions for determining a relative measure of (i) the number of at least 100 different SNPs in (a), and (ii) the number of SNPs for which heterozygosity is determined to be present, or the number of SNPs for which heterozygosity is determined to be absent, among the at least 100 different SNPs of (a) in a nucleic acid sample of, or derived from, genomic DNA of cancerous tissue of a subject of said species. In certain embodiments, the nucleic acid probes are attached to a solid or semi-solid phase.

According to certain aspects, the invention provides for a method for determining the probability of progression to cancer of pre-cancerous tissue in a subject comprising determining a GGDS for the precancerous tissue, wherein said GGDS is a relative measure of (a) number of heterozygous SNPs in a plurality of heterozygous SNPs, said plurality of heterozygous SNPs consisting of different SNPs wherein heterozygosity occurs in genomic DNA of non-cancerous tissue of said species to which said subject belongs, wherein said number of heterozygous SNPs in said plurality is in excess of 100 SNPs; and (b) the number of SNPs for which heterozygosity is determined to be present, or the number of SNPs for which heterozygosity is determined to be absent, among the number of heterozygous SNPs in said plurality of (a), in a nucleic acid sample of, or derived from, genomic DNA of precancerous tissue of the subject.

In certain embodiments, the invention provides for a computer comprising: a central processing unit; a memory, coupled to the central processing unit, the memory storing: (i) instructions for computing a GGDS for cancerous or precancerous tissue, wherein said GGDS is a relative measure of (a) number of heterozygous SNPs in a plurality of heterozygous SNPs, said plurality of heterozygous SNPs consisting of different SNPs wherein heterozygosity occurs in genomic DNA of non-cancerous tissue of said species to which said subject belongs, wherein said number of heterozygous SNPs in said plurality is in excess of 100 SNPs; and (b) the number of SNPs for which heterozygosity is determined to be present, or the number of SNPs for which heterozygosity is determined to be absent, among the number of heterozygous SNPs in said plurality of (a), in a nucleic acid sample of, or derived from, genomic DNA of cancerous or precancerous tissue of the subject. In certain embodiments, the memory further stores: (ii) instructions for comparing said GGDS to a threshold value; and (iii) instructions for outputing an indication of whether said GGDS is above or below a threshold value, or a phenotype based on said indication. In certain embodiments, the memory further stores in a database said number of heterozygous SNPs of (a). In certain embodiments, the memory further stores in a database an indication of the identity of each SNP in the heterozygous SNPs of (a). In certain embodiments, the number of heterozygous SNPs of (a) comprises heterozygous SNPs from noncancerous tissue of a plurality of members of said species, and wherein said identity of each heterozygous SNP in the database is associated with an identifier for which organism exhibits said heterozygous SNP. In certain embodiments, the memory further stores: (i) instructions for receiving SNP probe hybridization data; (ii) instructions for storing SNP probe hybridization data; (iii) instructions for comparing SNP probe hybridization data to determine whether an absence or presence of SNP heterozygosity has occurred in said nucleic acid sample from cancerous or precancerous tissue.

The invention also provides for a computer program product for use in conjunction with a computer system, the computer program product comprising a computer readable storage medium and a computer program mechanism embedded therein, the computer program mechanism comprising: (i) instructions for computing a GGDS for cancerous or precancerous tissue, wherein said GGDS is a relative measure of (a) number of heterozygous SNPs in a plurality of heterozygous SNPs, said plurality of heterozygous SNPs consisting of different SNPs wherein heterozygosity occurs in genomic DNA of non-cancerous tissue of said species to which said subject belongs, wherein said number of heterozygous SNPs in said plurality is in excess of 100 SNPs; and (b) the number of SNPs for which heterozygosity is determined to be present, or the number of SNPs for which heterozygosity is determined to be absent, among the number of heterozygous SNPs in said plurality of (a), in a nucleic acid sample of, or derived from, genomic DNA of cancerous or precancerous tissue of the subject. In certain embodiments, the computer program mechanism further comprises: (ii) instructions for comparing said GGDS to a threshold value; and (iii) instructions for outputing an indication of whether said GGDS is above or below a threshold value, or a phenotype based on said indication. In certain embodiments, the memory further stores in a database said number of heterozygous SNPs of (a). In certain embodiments, the memory further stores in a database an indication of the identity of each SNP in the heterozygous SNPs of (a). In certain embodiments, the number of heterozygous SNPs of (a) comprises heterozygous SNPs from noncancerous tissue of a plurality of members of said species, and wherein said identity of each heterozygous SNP in the database is associated with an identifier for which organism exhibits said heterozygous SNP. In certain embodiments, the memory further stores: (i) instructions for receiving SNP probe hybridization data; (ii) instructions for storing SNP probe hybridization data; (iii) instructions for comparing SNP probe hybridization data to determine whether an absence or presence of SNP heterozygosity has occurred in said nucleic acid sample from cancerous or precancerous tissue.

### Terminoloy

“Heterozygous SNP” means a SNP wherein the nucleotide at the position of the polymorphism differs (i.e., is a different nucleotide) in genomic DNA of a species, indicating that the nucleotide differs between two different alleles at a given locus on a pair of homologous chromosomes.

The term “about” means ±10% of the value the term to which the term is applied, or, if the foregoing is inapplicable, within standard experimental deviation.

### BRIEF DESCRIPTION OF THE DRAWINGS

FIG. 1 illustrates an exemplary embodiment of a computer system useful for implementing certain methods of this invention.

FIG. 2A-2D shows Kaplan-Meier survival curves for subjects with lung cancer for whom GGDS was determined. The x-axes show time in months and the y-axes show either the percent overall survival (OS) of patients or the percent disease-free survival (DFS) of patients. FIG. 2A (OS) and FIG. 2B (DFS) show survival for patients with low GGDS (<0.049) and high GGDS (>0.049). FIG. 2C (OS) shows survival for patients when the cohort was divided into quartiles of 11 patients each. The GGDS of each quartile are as follows: group 1: 0.003-0.0151; group 2: 0.0285-0.0483; group 3: 0.0503-0.0889; and group 4:0.0911-0.2043. FIG. 2D (OS) shows survival for patients when the cohort was divided into quartiles using the optimal GGDS threshold value of 0.041.

### DETAILED DESCRIPTION

The present invention relates to a method for determining phenotype of a cancer in a subject comprising determining global genome damage score (GGDS) for the cancer, wherein said GGDS is a relative measure of: (a) number of heterozygous SNPs in a plurality of heterozygous SNPs, said plurality of heterozygous SNPs consisting of different SNPs wherein heterozygosity occurs in genomic DNA of non-cancerous tissue (i.e., tissue that is believed to be free of cancer) of said species to which said subject belongs, wherein said number of heterozygous SNPs in said plurality is in excess of 100 SNPs; and (b) the number of SNPs for which heterozygosity is determined to be present, or the number of SNPs for which heterozygosity is determined to be absent, among the number of heterozygous SNPs in said plurality of (a), in a nucleic acid sample of, or derived from, genomic DNA of cancerous tissue of the subject.

“(a)” and “(b)” will be used hereinbelow to refer to elements (a) and (b), as defined in the above paragraph.

The phenotype of a cancer determined by the methods of the invention can be, for example, the predicted probability of survival, the predicted response to therapy, the predicted probability of metastasis, or the stage of cancer.

The present invention relates to a method for determining the probability of progression to cancer of pre-cancerous tissue in a subject comprising determining a GGDS for the precancerous tissue, wherein said GGDS is a relative measure of (a); and (b) wherein the nucleic acid sample is of, or derived from, genomic DNA of precancerous tissue of the subject instead of cancerous tissue.

The present invention also relates to computers and computer program products for practicing the methods of the invention.

### Determining Global Genome Damage Score

According to one aspect of the invention, global genome damage score is a relative measure determined by dividing the number of SNPs with loss of heterozygosity identified in the genomic nucleic acid from cancerous sample from a subject by the number of a plurality herterozygous SNPs (i.e., informative SNPs) identified in the genomic nucleic acid sample from non-cancerous tissue and/or cells of said species to which said subject belongs. For example, GGDS is a relative measure calculated by the number of SNPs for which heterozygosity is determined to be present, or the number of SNPs for which heterozygosity is determined to be absent in a nucleic acid sample from cancerous tissue, divided by the number of heterozygous SNPs in a plurality of SNPs wherein heterozygosity occurs in genomic DNA of non-cancerous tissue of said species to which said subject belongs. In certain embodiments, the number of SNPs with loss of heterozygosity identified in the nucleic acid from cancerous sample from a subject is measured by directly recording the number of SNPs exhibiting homozygosity. In certain embodiments, the number of SNPs with loss of heterozygosity identified in the nucleic acid from cancerous sample from a subject is measured by recording the number of SNPs exhibiting heterozygosity and subtracting from the total number of informative SNPs to determine the number of SNPs with loss of heterozygosity in the nucleic acid from a cancerous sample.

The GGDS is a relative measure of (a) and (b) (as described in Section 5 hereinabove). The GGDS can be expressed for example as the ratio of (a):(b) or (b):(a) or the logarithm of either ratio. The GGDS can be characterized by any convenient metric, e.g., arithmetic difference, ratio, log(ratio), etc. The mathematical operation log can be any logarithmic operation. In certain embodiments, it is the natural log or log10. As will be clear, the value of (b) used to compute GGDS can be the number of those heterozygous SNPs for which heterozygosity is maintained in the cancerous tissue of the subject or, in an alternative embodiment, the value of (b) used to compute GGDS can be the number of those heterozygous SNPs for which heterozygosity is lost in the cancerous tissue of the subject.

In the methods of the invention, SNPs are used in determining the phenotype of a cancer. There are six possible SNP types, either transitions (A<>T or G<>C) or transversions (A<>G, A<>C, G<>T or C<>T). SNPs are advantageous in that large numbers can be identified and scored for heterozygosity or absence of heterozygosity.

The invention provides methods for determining and/or predicting the phenotype of a cancer that involve determination of a GGDS in a subject. To determine the GGDS of a cancer in a subject, heterozygous SNPs are identified located throughout the genome using nucleic acid samples derived from non-cancerous tissue of the subject or a population of subjects of a single species, and the number is determined of those heterozygous SNPs identified that maintain heterozygosity (or alternatively do not exhibit heterozygosity, i.e., have lost heterozygosity) in a nucleic acid sample of, or derived from, genomic DNA of cancerous tissue of the subject. A nucleic acid sample “derived from” genomic DNA includes but is not limited to pre-messenger RNA (containing introns), amplification products of genomic DNA or pre-messenger RNA, fragments of genomic DNA optionally with adapter oligonucleotides ligated thereto or present in cloning or other vectors, etc. (introns and noncoding regions should not be selectively removed).

All of the SNPs known to exhibit heterozygosity in the species to which the subject with cancer belongs, need not be included in the number of heterozygous SNPs in (a). At a minimum, (a) should consist of at least (i.e., comprise) more than 100 such heterozygous SNPs. In specific embodiments, (a) consists of more than 500, 1,000, 1,500, 2,000, 2,500, 3,000, or 3,500 heterozygous SNPs. Preferably, such SNPs are in the human genome. In a specific embodiment, the plurality of heterozygous SNPs of (a) comprises SNPs comprising a nucleotide sequence complementary to the genomic DNA sequences of at least 100, 200, 300, 500, 1000, 1500, or 2000 different loci in the species to which the subject having cancer belongs. In a specific embodiment, the plurality of heterozygous SNPs of (a) comprises at least 100, 500, 1,000, 1,500, 2000, 2500, or 3000 SNPs that are randomly distributed throughout the genome at least every 250, 500, 1,000, 1,500, 2,000, 2,500, 3,000, or 5,000 kb pairs. By “randomly distributed,” as used above, is meant that the SNPs of the plurality are not selected by bias toward any specific chromosomal locus or loci; however, other biases (e.g., the avoidance of repetitive DNA sequences) can be used in the selection of the SNPs. In a specific embodiment, the plurality of heterozygous SNPs of (a) comprises at least 100, 500, 1,000, 1,500, 2,000, 2,500, or 3,000 SNPs that are not within the same 250, 500, 1,000, 1,500, or 2,000 kb region of genomic DNA as any other SNPs within the plurality. In a specific embodiment, the plurality of heterozygous SNPs of (a) is not found in regions of genomic DNA that are repetitive. In another specific embodiment, the plurality of heterozygous SNPs of (a) comprises SNPs located in the genome on different chromosomal loci, respectively, wherein the different chromosomal loci comprise loci on each of the chromosomes of the species, or on each arm of each chromosome of the species.

The heterozygous SNPs used in the methods of the invention to determine the phenotype of a cancer are informative, meaning heterozygosity is observed in the nucleic acid sample from non-cancerous tissue and/or cells of a subject. According to the methods of the invention for determining and/or predicting phenotype of a cancer, these informative SNPs are examined in the nucleic acid sample from a cancerous tissue and/or cells of a subject to determine presence or absence of heterozygosity which is then used to determine GGDS.

In certain embodiments, at least about 100, 200, 300, 400, 500, 600, 700, 800, 900, 1000, 1100, 1200, 1300, 1400, 1500, 1600, 1700, 1800, 1900,2000,2100,2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700,4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10,000, 10,100, 10,200, 10,300, 10,400, 10,500, 10,600, 10,700, 10,800, 10,900, 11,000, 11,100, 11,200, 11,300, 11,400, 11,500, 11,600, 11,700, 11,800, 11,900, 12,000, 12,100, 12,200, 12,300, 12,400, 12,500, 12,600, 12,700, 12,800, 12,900, 13,000, 13,100, 13,200, 13,300, 13,400, 13,500, 13,600, 13,700, 13,800, 13,900, 14,000, 14,100, 14,200, 14,300, 14,400, 14,500, 14,600, 14,700, 14,800, 14,900, or 15,000 SNPs are examined in nucleic acid samples derived from noncancerous tissue to identify informative heterozygous SNPs (all or a subset of which can constitute (a) as described in Section 5 above). In certain embodiments, about 100 to 500, 250 to 750, 500 to 1,000, 750 to 1,250, 1,000 to 1,500, 1,250 to 1,750, 1,500 to 2,000, 1,750 to 2,250, 2,000 to 2,500, 2,250 to 2,750, 2,500 to 3,000, 2,750 to 3,250, 3,000 to 3,500, 3,250 to 3,750, 3,500 to 4,000, 3,750 to 4,250, 4,000 to 4,500, 4,250 to 4,750, 4,500 to 5,000, 4,750 to 5,250, 5,000 to 5,500, 5,250 to 5,750, 5,500 to 6,000, 5,750 to 6,250, 6,000 to 6,500, 6,250 to 6,750, 6,500 to 7,000, 6,750 to 7,250, 7,000 to 7,500, 7,250 to 7,750, 7,500, to 8,000, 7,750 to 8,250, 8,000 to 8,500, 8,250 to 8,750, 8,500 to 9,000, 8,750 to 9,250, 9,000 to 9,500, 9,250, to 9,750, 9,500 to 10,000, 9,750 to 10,250, 10,000 to 10,500, 10,250 to 10,750, 10,500 to 11,000, 10,750 to 11,250, 11,000 tol 1,500, 11,250 to 11,750, 11,500 to 12,000, 11,750 to 12,250, 12,000 to 12,500, 12,250 to 12,750,12,500 to 13,000, 12,750 to 13,250, 13,000 to 13,500, 13,250 to 13,750, 13,500 to 14,000, 13,750 to 14,250, 14,000 to 14,500, 14,250 to 14,750, 14,500 to 15,000, or 14,750 to 15,250 SNPs are examined in nucleic acid samples derived from noncancerous tissue to identify informative heterozygous SNPs (all or a subset of which can constitute (a)).

In a specific embodiment, the nucleic acid samples used to determine the value of (a) that can be used to compute GGDS, that is, the number of heterozygous SNPs in the plurality of SNPs, that exhibit heterozygosity in genomic DNA of non-cancerous tissue of the species to which the cancer patient belongs, are taken from at least 1, 2, 5, 10, 20, 30, 40, 50, 100, or 250 different organisms of that species.

In a specific embodiment, where the value for (a) is not known it can be determined (e.g., by using a SNP array with at least 100, 500, 1000, 5000, or 10,000 SNP probes, (e.g., those sold by Affymetrix, Santa Clara, Calif.)) among which the SNPs that exhibit heterozygosity in noncancerous tissue can be determined. (a) can be all or a subset of such determined SNPs.

Briefly, a plurality of SNPs that exhibit heterozygosity in non-cancerous tissue can be determined in the species of interest by collecting genomic nucleic acid from noncancerous cells of organism(s) of the same species as the subject, or from the subject. The genomic nucleic acid or nucleic acid derived therefrom (e.g., by restriction digestion, amplification or genome-wide cloning; or pre-RNA) from noncancerous cells is isolated. In certain embodiments, the genomic nucleic acid is digested with restriction enzymes and/or amplified. The nucleic acid samples are hybridized to SNP probes to identify heterozygous SNPs genome-wide. (a) can be all or a portion of such identified SNPs.

The value for (b) is also determined. The genomic nucleic acid from cancerous cells is isolated and can be digested with restriction enzymes and/or amplified. SNP locus heterozygosity in the nucleic acid from cancer cells at the heterozygous loci identified in the nucleic acid from noncancerous cells is then measured. Sections 5.9 through 5.13 provide a detailed description of exemplary methods for determination of heterozygosity that can be used in the methods of the invention for determining and/or predicting the phenotype of a cancer.

In certain embodiments, at least 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10,000, 10,100, 10,200, 10,300, 10,400, 10,500, 10,600, 10, 700, 10,800, 10,900, 11,000, 11,100, 11, 200, 11,300, 11,400, 11,500, 11,600, 11,700, 11,800, 11,900, 12,000, 12,100, 12,200, 12,300, 12,400, 12,500, 12,600, 12,700, 12,800, 12,900, 13,000, 13,100, 13,200, 13,300, 13,400, 13,500, 13,600, 13,700, 13,800, 13,900, 14,000, 14,100, 14,200, 14,300, 14,400, 14,500, 14,600, 14,700, 14,800, 14,900, or 15,000 informative SNPs are used in the methods of the invention, i.e. to constitute (a), and their heterozygosity is queried to determine (b). In preferred embodiments, about 100 to 6000 informative SNPs are used in such methods of the invention.

In certain embodiments, the informative SNPs of (a) used in the methods of the invention to determine and/or predict the phenotype of a cancer are not located in regions of the subjects genome characterized by repetitive DNA. In certain embodiments, about 10%, 20%, 30%, 40%, 50%, 60%, 70% 80%, 90% or more of the region (i.e. within about 500 KB of the SNP) may comprise repetitive genomic DNA. Typically, repetitive DNA comprises tandem repeats of segments of DNA. Such segments can be, for example, 2, 3, 4, 5, 6, 7, 8, 9, 10, 11, 12, 13, or 15 bp in length. The segments may be repeated 5, 10, 15, 20, 25, 30, 35, 40, 45, 50 times, or more. This repetitive DNA allows for hybridization at a SNPs of nucleic acid fragments not corresponding to the SNPs, resulting in a decrease in hybridization specificity and decrease in resolution of a hybridization readout. In specific embodiments, where SNPs used in the methods of the invention are located in regions of repetitive genomic DNA, the oligonucleotide SNP probes used to identify informative SNPs should be at least 20 bp, 22 bp, 24 bp, 26 bp, 28 bp, 30 bp, 32 bp, 34 bp, 36 bp, 38 bp, 40 bp, 42 bp, 44 bp, 46 bp, 48 bp, 50 bp, 52 bp, 54 bp, 56 bp, 58 bp, or 60 bp in length.

In certain embodiments, the informative SNPs of (a) used in the methods of the invention to determine and/or predict the phenotype of a cancer comprise at least one SNP on each chromosome of a subject. In a related embodiment, the informative SNPs used in the methods of the invention to determine and/or predict the phenotype of a cancer comprise at least one SNP on each arm of each chromosome of a subject.

In preferred embodiments, the informative SNPs of (a) used in the methods of the invention to determine and/or predict the phenotype of a cancer comprise at least one SNP on each of the 23 pairs of human chromosomes. In preferred embodiments, the informative SNPs of (a) used in the methods of the invention to determine and/or predict the phenotype of a cancer comprise at least one SNP on each arm of each the 23 pairs of human chromosomes. In preferred embodiments, the informative SNPs used in the methods of the invention to determine and/or predict the phenotype of a cancer comprise at least two SNPs on each arm of each the 23 pairs of human chromosomes.

In certain embodiments, the informative SNPs of (a) used in the methods of the invention to determine and/or predict the phenotype of a cancer are distributed throughout the genome of a subject. For example, there may be at least one informative SNP at least every 500 kb, 400 kb, 300 kb, 200 kb, 100 kb, 50 kb, 40 kb, 30 kb, 20 kb, 10 kb throughout the genome of a human subject. In certain embodiments, SNPs of (a) are distributed throughout the genome of a subject where two SNPs have an average separation of at least 500 kb, 400 kb, 300 kb, 200 k, 100 kb, 50 kb, 40 kb, 30 kb, 20 kb, 10 kb or less.

### Prediction of Survival

In certain embodiments, the invention provides methods for determining the phenotype of a cancer wherein the phenotype is survival of the subject having cancer. In such embodiments, the GGDS is a measure of the survival for a subject. The phenotype determined and/or predicted can be overall survival or disease-free survival. Overall survival preferably is measured from the date of diagnosis to the date of death. Disease-free survival preferably is measured from the date of surgical removal of cancerous tissue to the date of disease recurrence.

Where GGDS represents loss of heterozygosity (i.e., where the value of (b) described above used to compute the GGDS is the number of SNPs for which heterozygosity is determined to be absent (lost)), subjects whose cancerous tissue exhibits a GGDS below a threshold value are predicted to live longer and have disease recurrence later than those with high GGDS (above the threshold value).

Where GGDS represents retention of heterozygosity (i.e., where the value of (b) described above used to compute the GGDS is the number of SNPs for which heterozygosity is determined to be present), subjects whose cancerous tissue exhibits a GGDS above a threshold value are predicted to live longer and have disease recurrence later than those with low GGDS (below the threshold value). [As will be clear, in such an embodiment and other embodiments described throughout the specification, where the value of (b) used to compute GGDS is the number of SNPs for which heterozygosity is determined to be present, predictions based on GGDS's being above threshold values are switched to when GGDS's are below threshold values, and vise vera.]

For example, once a GGDS has been determined for a population of subjects, overall survival and/or disease-free survival can be monitored over a period of time for the population in order to determine appropriate threshold values. In preferred embodiments, the survival values used in the methods of the invention are determined from death and recurrence data recorded over a period of up to about 200 months. The period of time for which subjects are monitored can vary. For example, subjects may be monitored for at least 2, 4, 6, 8, 10, 12, 14, 16, 18, 20, 25, 30, 35, 40, 45, 50, 55, or 60 months, or up to any of these time periods. GGDS threshold values that correlate to survival can be determined, for example, as described in the Example section below (see section 6). By way of example, Kaplan-Meier survival curves can be plotted as described in the Example section below to identify or confirm GGDS threshold values that correlate to survival. Kaplan-Meier survival curves can provide a long-term estimate of survival based on short-term data from clinical studies. In certain embodiments, subjects with GGDS values at or below the threshold value (where the value of (b) described above used to compute the GGDS is the number of SNPs for which heterozygosity was determined to be absent (lost), rather than the alternative embodiment where (b) is the number of SNPs for which heterozygosity was determined to be present) exhibit an overall survival or disease-free survival probability that is at least a 50%, 55%, 60%, 65%, 70%, 75%, 80%, 85%, 90%, 95%, or 100% probability of survival within a given time period. In certain embodiments, the probability of survival is for at least 2 years, 4 years, 6 years, 8 years, 10 years, 12 years, 14 years, 15 years, or more.

In a specific embodiment, the threshold level for human subjects with non-small cell lung carcinoma is a GGDS of 0.041, and patients with GGDS (with (b) being the number of SNPs for which heterozygosity is lost) at or below 0.041 are predicted to live longer and have disease recurrence later than those with high GGDS. By way of explanation, but without being bound by any particular mechanism, it is believed that cancerous tissue exhibiting a GGDS below such a threshold (with less loss of heterozygosity) has a high capacity for DNA repair, resulting in longer survival (and less metastasis).

### Prediction of Response to Therapy

In certain embodiments, the invention provides methods for determining the phenotype of a cancer wherein the phenotype is response to therapy. The therapy may be any anti-cancer therapy including, but not limited to, chemotherapy, radiation therapy, and immunotherapy (see Section 5.3.1).

The outcome of therapy for a cancer can be determined and/or predicted using the methods of the invention. In such embodiments, the GGDS is predictive of the outcome of anti-cancer therapy for a subject.

Where GGDS represents loss of heterozygosity (i.e., where the value of (b) described above used to compute the GGDS is the number of SNPs for which heterozygosity is determined to be absent (lost)), subjects whose cancerous tissue exhibits a GGDS below a threshold value are predicted to have a poorer response to therapy (e.g., radiation or chemotherapy) than those with high GGDS (above the threshold value).

Where GGDS represents retention of heterozygosity (i.e., where the value of (b) described above used to compute the GGDS is the number of SNPs for which heterozygosity is determined to be present), subjects whose cancerous tissue exhibits a GGDS above a threshold value are predicted to have a poorer response to therapy (e.g., radiation or chemotherapy) than those with low GGDS (below the threshold value).

For example, in order to determine appropriate threshold values, a particular anti-cancer therapeutic regimen can be administered to a population of subjects and the outcome can be correlated to GGDS's that were determined prior to administration of any anti-cancer therapy. Overall survival and disease-free survival can be monitored over a period of time for subjects following anti-cancer therapy for whom GGDS values are known. In certain embodiments, the same doses of anti-cancer agents are administered to each subject. In related embodiments, the doses administered are standard doses known in the art for anti-cancer agents. The period of time of which subjects are monitored can vary. For example, subjects may be monitored for at least 2, 4, 6, 8, 10, 12, 14, 16, 18, 20, 25, 30, 35, 40, 45, 50, 55, or 60 months. GGDS threshold values that correlate to outcome of an anti-cancer therapy can be determined using methods such as those described in the Example section for overall survival and disease-free survival. By way of example, Kaplan-Meier survival curves can be plotted as described in the Example section below to identify or confirm GGDS threshold values that correlate to outcome of a therapy. Kaplan-Meier survival curves can provide a long-term estimate of survival based on short-term data from clinical studies. In certain embodiments, subjects with GGDS values at or below the threshold value are predicted to exhibit an overall survival or disease-free survival probability following anti-cancer therapy that is at least 50%, 55%, 60%, 65%, 70%, 75%, 80%, 85%, 90%, 95%, or 100% within a given time period. In certain embodiments, the probability of survival following anti-cancer therapy is for at least 2 years, 4 years, 6 years, 8 years, 10 years, 12 years, 14 years, 15 years or more.

By way of explanation, but without being bound by any particular mechanism, it is believed that a high GGDS value (where the value of (b) used to compute GGDS is the number of SNPs for which heterozygosity is determined to be absent), while a predictor of poor survival, might indicate that a subject's DNA repair mechanisms are impaired or overwhelmed. In such subjects, anti-cancer therapies that cause damage to DNA are predicted to have greater efficacy because cancerous cells damaged by such therapy would not repair the damage and thus would undergo cell death. However, because the subjects' DNA repair mechanism is impaired, anti-cancer therapies that damage DNA are believed to result in intensified side effects, or a worsening of the overall health of a subject because DNA from non-cancerous tissues is also repaired less effectively. For certain subjects, such considerations may outweigh any potential benefits of chemotherapy or radiation therapy. In such instances, it may be preferable to use a non-chemotherapeutic approach such as, but not limited to, surgery to remove cancerous tissue.

In contrast, low GGDS values (where the value of (b) used to compute GGDS is the number of SNPs for which heterozygosity is determined to be absent) in subjects are believed to be positive predictors of survival; however, such subjects are believed to have a greater capacity for DNA repair in comparison to subjects with high GGDSs. In such subjects, anti-cancer therapies that cause damage to DNA are predicted to have less efficacy because cancerous cells damaged by such therapy have higher capacities for repairing DNA, resulting in survival of the cancerous cells. Because the capacity for DNA repair is high in non-cancerous cells or tissues, subjects with low GGDS would have fewer side effects from anti-cancer therapies that damage DNA.

Thus, in clinical practice, accurate prognosis of cancer phenotype according to the present invention, including determination of survival and/or outcome of therapy, could allow the oncologist to tailor the administration of therapy to a subject.

### Anti-Cancer Therapeutic Agents

Anti-cancer therapies which damage DNA such as chemotherapy or radiation therapy are predicted to have efficacy in subjects determined to have high GGDS (where the value of (b) used to compute GGDS is the number of SNPs for which heterozygosity is determined to be absent) using the methods of the invention for determining the phenotype of a cancer.

Chemotherapy includes the administration of a chemotherapeutic agent. Such a chemotherapeutic agent can be, but is not limited to, one selected from among the following groups of compounds: cytotoxic antibiotics, antimetabolities, anti-mitotic agents, alkylating agents, platinum compounds, arsenic compounds, DNA topoisomerase inhibitors, taxanes, nucleoside analogues, plant alkaloids, and toxins; and synthetic derivatives thereof. Exemplary compounds of the groups include, but are not limited to, alkylating agents: treosulfan, trofosfamide, and cisplatin; plant alkaloids: vinblastine, paclitaxel, docetaxol; dna topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. The foregoing examples of chemotherapeutic agents is illustrative, and is not intended to be limiting.

The radiation used in radiation therapy can be ionizing radiation. Radiation therapy can also be gamma rays or X-rays. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

Anti-cancer therapies which damage DNA to a lesser extent than chemotherapy or radiation therapy may have efficacy in subjects determined to have low GGDS (where the value of (b) used to compute GGDS is the number of SNPs for which heterozygosity is determined to be absent) using the methods of the invention for determining the phenotype of a cancer. Examples of such therapies include immunotherapy, hormone therapy, and gene therapy.

Gene therapy can be conducted using methods such as, but not limited to, antisense polynucleotides, ribozymes, RNA interference molecules, triple helix polynucleotides and the like, where the nucleotide sequence of such compounds are related to the nucleotide sequences of DNA and/or RNA of genes that are linked to the initiation, progression, and/or pathology of a tumor or cancer. For example, many are oncogenes, growth factor genes, growth factor receptor genes, cell cycle genes, DNA repair genes, and are well known in the art.

Immunotherapy may comprise, for example, use of cancer vaccines and/or sensitized antigen presenting cells. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines.

Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In one embodiment, anti-cancer therapy used for cancers whose phenotype is determined by the methods of the invention can comprise one or more types of therapies described herein including, but not limited to, chemotherapeutic agents, immunotherapeutics, anti-angiogenic agents, cytokines, hormones, antibodies, polynucleotides, radiation and photodynamic therapeutic agents. For example, combination therapies can comprise one or more chemotherapeutic agents and radiation, one or more chemotherapeutic agents and immunotherapy, or one or more chemotherapeutic agents, radiation and chemotherapy.

The duration of treatment with anti-cancer therapies may vary according to the particular anti-cancer agent or combination thereof used. An appropriate treatment time for a particular cancer therapeutic agent will be appreciated by the skilled artisan. The invention contemplates the continued assessment of optimal treatment schedules for each cancer therapeutic agent, where the phenotype of the cancer of the subject as determined by the methods of the invention is a factor in determining optimal treatment doses and schedules.

### Prediction of Metastasis

In certain embodiments, the invention provides methods for determining the phenotype of a cancer wherein the phenotype is metastasis. In embodiments of the invention wherein metastasis is determined and/or predicted using the methods of the invention the subject is in an early, i.e., pre-metastasis, stage of a cancer. In such embodiments, the GGDS is a predictive measure of metastasis.

According to certain aspects of the present invention, likelihood of and/or time to metastasis of a cancer can be predicted using the methods of the invention in subjects having a cancer that has not yet metastasized.

Where GGDS represents loss of heterozygosity (i.e., where the value of (b) described above used to compute the GGDS is the number of SNPs for which heterozygosity is determined to be absent (lost)), subjects whose cancerous tissue exhibits a GGDS below a threshold value are predicted to have less likelihood of metastasis within a defined time period (the time period being dependent on the cancer type, e.g., 1 year, 2 years, 5 years, or 10 years) than those with high GGDS (above the threshold value).

Where GGDS represents retention of heterozygosity (i.e., where the value of (b) described above used to compute the GGDS is the number of SNPs for which heterozygosity is determined to be present), subjects whose cancerous tissue exhibits a GGDS above a threshold value are predicted to have less likelihood of metastasis within a defined time period (the time period being dependent on the cancer type, e.g., 1 year, 2 years, 5 years, or 10 years) than those with low GGDS (below the threshold value).

For example, to determine appropriate threshold values, the outcome of a population of subjects with pre-metastasis cancer can be correlated to GGDS's that were determined prior to clinical diagnosis of any metastasis. Metastasis can be monitored over a period of time for subjects for whom GGDS values are known. Metastasis can be monitored by methods well known in the clinical cancer art including, but not limited to, detection of cancerous cells in blood and lymph tissues or biopsy. The period of time of which subjects are monitored can vary. For example, subjects can be monitored for at least 2, 4, 6, 8, 10, 12, 14, 16, 18, 20, 25, 30, 35, 40, 45, 50, 55, or 60 months. GGDS threshold values that correlate to outcome of metastasis can be determined using methods such as those described in the Example section for overall survival and disease-free survival. Kaplan-Meier survival curves can be plotted as described in the Example section below to identify or confirm GGDS threshold values that correlate to metastasis. Kaplan-Meier survival curves can provide a long-term estimate of survival based on short-term data from clinical studies. In certain embodiments, for subjects with GGDS values at or below the determined threshold value, the probability of remaining free of metastasis is predicted to be at least 50%, 55%, 60%, 65%, 70%, 75%, 80%, 85%, 90%, 95%, or 100% within a given time period. In certain embodiments, the probability of remaining free of metastasis is for at least 2 years, 4 years, 6 years, 8 years, 10 years, 12 years, 14 years, 15 years or more. In certain embodiments, the clinical history of a subject can be used. For example, data from short-term clinical studies can be used to generate Kaplan-Meier survival curves to estimate the long-term probability of recurrence. This enables monitoring of subjects for a shorter period of time to determine threshold GGDS values. In certain embodiments, the percent probability of remaining free of metastasis or of developing metastasis for subjects with GGDS values above and/or below the determined threshold value can be extrapolated for up to about 20 months, 30 months, 40 months, 50 months, 60 months, 70 months, 80 months, 90 months, 100 months, 110 months, 120 months, 140 months, 150 months, 160 months, 170 months, or 200 months. In preferred embodiments, the estimations are extrapolated for up to 140 months. Thus, the present methods of the present invention for predicting metastasis provide an prognosis tool that is independent of, and can be used in conjunction with or in addition to, the traditional clinical prognosis model of the stages of progression of cancer described below.

The progression of cancer is typically characterized by the degree to which the cancer has spread through the body and is often broken into the following four stages. Stage I: The cancer is localized to a particular tissue such as, but not limited to, the lung or breast, and has not spread to the lymph nodes. Stage II: The cancer has spread to the nearby lymph nodes, i.e., metastasis. Stage III: The cancer is found in the lymph nodes in regions of the body away from the tissue of origin and may comprise a mass or multiple tumors as opposed to one. Stage IV: The cancer has spread to a distant part of the body. The stage of a cancer can be determined by clinical observations and testing methods that are well known to those of skill in the art. The stages of cancer model described above are traditionally used in conjunction with clinical diagnosis, and can be used in conjunction with the methods of the present invention, to predict the future development of a cancer and likelihood of success in therapy.

### Prediction of Recurrence

In certain embodiments, the invention provides methods for determining the phenotype of a cancer wherein the phenotype is probability of recurrence of cancer following treatment. In such embodiments, the GGDS is a predictive measure of cancer recurrence for a subject. The recurrence of the cancer following treatment can be in the tissue of origin or in another part of the subject's body. Treatment includes, but is not limited to, surgical removal of a cancer and/or anti-cancer therapies such as those described in Section 5.3.1.

Since the phenotype determined and/or predicted can be disease-free survival, which in a specific embodiment, is measured from the date of surgical removal of cancerous tissue to the date of disease recurrence, the above description for determining and/or predicting disease-free survival is applicable to determining and/or predicting recurrence of cancer (see Section 5.2). In embodiments of the methods of the invention wherein recurrence is predicted for subjects having had treatment comprising therapy with an anti-cancer agent, the above description for determining and/or predicting survival following therapy is applicable to determining and/or predicting recurrence of cancer (see Section 5.3). In such embodiments, recurrence can be observed and recorded in a population of subjects over time to determine a threshold GGDS values that are predictive of recurrence. To make this determination subjects can be monitored for up to about 2, 4, 6, 8, 10, 15, 20, 25, 30, 35, 40, 45, 50, 55, 60, 65, or 70 months following removal of the cancer or anti-cancer therapy.

In certain embodiments, the clinical history of a subject can be used. For example, data from short-term clinical studies can be used to generate Kaplan-Meier survival curves to estimate the long-term probability of recurrence. This enables monitoring of subjects for a shorter period of time to determine threshold GGDS values.

### Cancers for which Phenotype can be Determined

The methods of the invention can be used to determine the phenotype of different cancers. Specific examples of types of cancers for which the phenotype can be determined by the methods encompassed by the invention include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

In preferred embodiments, the cancer whose phenotype is determined by the method of the invention is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In preferred embodiments, the cancer is breast cancer, prostrate cancer, lung cancer, or colon cancer. In certain embodiments, the epithelial cancer is non-small-cell lung cancer, nonpapillary renal cell carcinoma, cervical carcinoma, ovarian carcinoma, or breast carcinoma. The epithelial cancers may be characterized in various other ways including, but not limited to, serous, endometrioid, mucinous, clear cell, brenner, or undifferentiated.

### Determination of Risk of Progression from a Precancerous to a Cancerous Condition

In related embodiments, the methods of the invention as described herein for prediction of phenotype of a cancer and for determining GGDS can be carried out as described, except using samples derived from precancerous tissue instead of cancerous tissue, to predict the phenotype of precanerous tissue, e.g., the probability of progression of the precancerous tissue to cancer.

Where GGDS represents loss of heterozygosity (i.e., where the value of (b) described above used to compute the GGDS is the number of SNPs for which heterozygosity is determined to be absent (lost)), subjects whose precancerous tissue exhibits a GGDS below a threshold value are predicted to have less likelihood of progression of the precancerous tissue to cancer within a defined time period (the time period being dependent on the potential cancer type, e.g., 1 year, 2 years, 5 years, or 10 years) than those with high GGDS (above the threshold value).

Where GGDS represents retention of heterozygosity (i.e., where the value of (b) described above used to compute the GGDS is the number of SNPs for which heterozygosity is determined to be present), subjects whose precancerous tissue exhibits a GGDS above a threshold value are predicted to have less likelihood of progression of the precancerous tissue to cancer within a defined time period (the time period being dependent on the potential cancer type, e.g., 1 year, 2 years, 5 years, or 10 years) than those with low GGDS (below the threshold value).

For example, to determine appropriate threshold values, the outcome of a population of subjects with precancerous tissue can be correlated to GGDS's that were determined prior to progression of a precancerous tissue to cancer. Progression can be monitored over a period of time for subjects for whom GGDS values are known. Progression can be monitored by methods well known in the clinical cancer art including, but not limited to, detection of precancerous and/or cancerous cells in tissue or blood samples. The period of time of which subjects are monitored can vary. For example, subjects can be monitored for at least 2, 4, 6, 8, 10, 12, 14, 16, 18, 20, 25, 30, 35, 40, 45, 50, 55, or 60 months. GGDS threshold values that correlate to progression to cancer can be determined using methods such as those described in the Example section. In certain embodiments, GGDS threshold values that correlate to progression to cancer can also be correlated to overall survival and disease-free survival where a population of subjects with precanceorus tissue is monitored through progression to cancer and through outcome of cancer. Kaplan-Meier survival curves can be plotted as described in the Example section below to identify or confirm GGDS threshold values that correlate to progression. Kaplan-Meier survival curves can provide a long-term estimate of progression or survival based on short-term data from clinical studies. In certain embodiments, for subjects with GGDS values at or below the determined threshold value, the probability of remaining free of progression to cancer is predicted to be at least 50%, 55%, 60%, 65%, 70%, 75%, 80%, 85%, 90%, 95%, or 100% within a given time period. In certain embodiments, the probability of remaining free of progression to cancer is for at least 2 years, 4 years, 6 years, 8 years, 10 years, 12 years, 14 years, 15 years or more. In certain embodiments, the percent probability of progression to cancer for subjects with GGDS values above and/or below the determined threshold value can be extrapolated for up to about 20 months, 30 months, 40 months, 50 months, 60 months, 70 months, 80 months, 90 months, 100 months, 110 months, 120 months, 140 months, 150 months, 160 months, 170 months, or 200 months.

In one embodiment, the invention provides for a method for determining the probability of progression to cancer of precancerous tissue in a subject comprising determining a GGDS for the precancerous tissue, wherein said GGDS is a relative measure of (a) number of heterozygous SNPs in a plurality of heterozygous SNPs, said plurality of heterozygous SNPs consisting of different SNPs wherein heterozygosity occurs in genomic DNA of non-cancerous tissue of said species to which said subject belongs, wherein said number of heterozygous SNPs in said plurality is in excess of 100 SNPs; and (b) the number of SNPs for which heterozygosity is determined to be present, or the number of SNPs for which heterozygosity is determined to be absent, among the number of heterozygous SNPs in said plurality of (a), in a nucleic acid sample of, or derived from, genomic DNA of precancerous tissue of the subject.

In embodiments of the invention where the probability of progression to cancer of a precancerous tissue is determined, the cancer can be any cancer such as, but not limited to those described above in Section 5.6 and is preferably an epithelial malignancy.

In specific embodiments of the invention where the probability of progression to cancer of a precancerous tissue is determined, the precancerous tissue can be: hyperplastic, dysplastic, or metaplastic tissue; tissue exposed to known carcinogens; tissue of a subject that was exposed to a carcinogen, chemotoxic agent, and/or radiation known to affect such tissue; or any other tissue believed to have and increased likelihood of development of cancer. Such exposure can be repeated and/or localized to a particular portion of a subject's body.

The threshold GGDS value can be determined using methods analogous to those described in Section 5.8.1 for subjects previously treated with chemotherapy or radiation.

Precancerous tissues that can be used in the invention include, for example, tissue that often progresses to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79.) Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the presence of precancerous tissue. Such characteristics of a transformed phenotype include morphology changes, looser substratum attachment, loss of contact inhibition, loss of anchorage dependence, protease release, increased sugar transport, decreased serum requirement, expression of fetal antigens, etc. (see also id., at pp. 84-90 for characteristics associated with a transformed or malignant phenotype).

Examples of precancerous tissues include, but are not limited to, leukoplakia, a benign-appearing hyperplastic or dysplastic lesion of the epithelium, or Bowen's disease, a carcinoma in situ, which are pre-neoplastic lesions; and fibrocystic disease (cystic hyperplasia, mammary dysplasia, particularly adenosis (benign epithelial hyperplasia)).

In other embodiments, a patient which exhibits one or more of the following predisposing factors for cancer in a tissue can be prognosed by the methods of the invention for the progression to cancer: a chromosomal translocation associated with a malignancy (e.g., the Philadelphia chromosome for chronic myelogenous leukemia, t(14;18) for follicular lymphoma, etc.), familial polyposis or Gardner's syndrome (possible forerunners of colon cancer), benign monoclonal gammopathy (a possible forerunner of multiple myeloma), and a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 1976, Basic Pathology, 2d Ed., W. B. Saunders Co., Philadelphia, pp. 112-113) etc.).

Thus, the present methods of the present invention for predicting progression of a precancerous tissue to cancer based on GGDS provide a prognostic tool that is independent of, and can be used in conjunction with or in addition to, the traditional clinical prognosis techniques described herein based on the phenotype of precancerous tissue.

### Subjects

In preferred embodiments, the subject for whom a phenotype of a cancer is determined using the methods of the invention, or for whom the risk of progression from a precancerous to a cancerous condition is determined, is a mammal (e.g., mouse, rat, primate, non-human mammal, domestic animal such as dog, cat, cow, horse), and is most preferably a human.

In preferred embodiments of the methods of the invention, the subject has not undergone chemotherapy or radiation therapy. In alternative embodiments, the subject has undergone chemotherapy or radiation. In related embodiments, the subject has not been exposed to levels of radiation or chemotoxic agents above those encountered generally or on average by the subjects of a species and wherein the levels are capable of causing significant damage to DNA.

In certain embodiments, the subject has had surgery to remove cancerous or precancerous tissue. In embodiments, where the cancerous tissue has not been removed, the cancerous tissue may be located in an inoperable region of the body, a tissue that is essential for life, or in a region where a surgical procedure would cause considerable risk of harm to the patient.

### Subjects Previously Treated with Chemotherapy or Radiation

According to one aspect of the invention, GGDS can be used to determine the phenotype of a cancer in a subject where the subject has previously undergone chemotherapy, radiation therapy, or has been exposed to radiation, or a chemotoxic agent. Such therapy or exposure could potentially damage DNA and alter the numbers of informative heterozygous SNPs in a subject. The altered number of informative heterozygous SNPs would in turn alter the GGDS of a subject. Because the non-cancerous DNA samples would exhibit greater or fewer heterozygous SNPs, the range of GGDSs would be altered for a population of subjects.

To determine GGDS threshold values for the various phenotypes of a cancer described above where the subjects exhibit DNA damage from therapy or exposure, a population of subjects monitored preferably has had chemotherapy or radiation therapy, preferably via identical or similar treatment regimens, including dose and frequency, for each subject.

The phenotype determined and/or predicted can be any of those described above. The methods described above are applicable to determining and/or predicting survival cancer (see Section 5.2), response to additional therapy (see Section 5.3), metastasis cancer (see Section 5.4), or recurrence of cancer (see Section 5.5). In embodiments of the methods of the invention where phenotype is determined and/or predicted for subjects having previously had DNA damage from therapy or exposure to a chemotoxic agent or radiation, the above described methods are altered in that the population of subjects used to determine predictive GGDS threshold values have all previously had DNA damage resulting from therapy or exposure. In certain embodiments, DNA damage from therapy or exposure in a subject or population of subjects occurs about 1 month, 2 months, 3 months, 4 months, 5 months, 6 months, 7 months, 8 months, 9 months, 10 months, 11 months, 1 year, 1.5 years, 2 years or more before determination of GGDS. Using populations of subjects with DNA damage from therapy or exposure, GGDS threshold values that are determinative and/or predictive of the phenotype of a cancer can be determined. Such threshold values can then be applied to subjects having cancer who have previous DNA damage from therapy or exposure to determine and/or predict a phenotype of the cancer.

### Nucleic Acid Sample Preparation

### Nucleic Acid Isolation

Nucleic acid samples derived from cancerous and non-cancerous cells of a subject that can be used in the methods of the invention to determine the phenotype of a cancer can be prepared by means well known in the art. For example, surgical procedures or needle biopsy aspiration can be used to collect cancerous samples from a subject. The cancerous tissue and/or cell samples can then be microdissected to reduce amount of normal tissue contamination prior to extraction of genomic nucleic acid or pre-RNA for use in the methods of the invention.

Collecting nucleic acid samples from non-cancerous cells of a subject can also be accomplished with surgery or aspiration. In surgical procedures where cancerous tissue is removed, surgeons often remove non-cancerous tissue and/or cell samples of the same tissue type of the cancer patient for comparison. Nucleic acid samples can be isolated from such non-cancerous tissue of the subject for use in the methods of the invention.

In certain embodiments of the methods of the invention, nucleic acid samples from non-cancerous tissues are not derived from the same tissue type as the cancerous tissue and/or cells sampled, and/or are not derived from the cancer patient. The nucleic acid samples from non-cancerous tissues may be derived from any non-cancerous and/or disease-free tissue and/or cells. Such non-cancerous samples can be collected by surgical or non-surgical procedures. In certain embodiments, non-cancerous nucleic acid samples are derived from tumor-free tissues. For example, non-cancerous samples may be collected from lymph nodes, peripheral blood lymphocytes, and/or mononuclear blood cells, or any subpopulation thereof. In a preferred embodiment, the noncancerous tissue is not precancerous tissue, e.g., it does not exhibit any indicia of a pre-neoplastic condition such as hyperplasia, metaplasia, or dysplasia.

In a specific embodiment, the nucleic acid samples used to determine the values of (a) used to compute GGDS, that is, the number of heterozygous SNPs in the plurality of SNPs, that exhibit heterozygosity in genomic DNA of non-cancerous tissue of the species to which the cancer patient belongs, are taken from at least 1, 2, 5, 10, 20, 30, 40, 50, 100, or 200 different organisms of that species.

According to certain aspects of the invention, nucleic acid “derived from” genomic DNA, as used in the methods of the invention, e.g., in hybridization experiments to determine heterozygosity of SNPs, can be fragments of genomic nucleic acid generated by restriction enzyme digestion and/or ligation to other nucleic acid, and/or amplification products of genomic nucleic acids, or pre-messenger RNA (pre-mRNA), amplification products of pre-mRNA, or genomic DNA fragments grown up in cloning vectors generated, e.g., by “shotgun” cloning methods. In certain embodiments, genomic nucleic acid samples are digested with restriction enzymes. In preferred embodiments, the nucleic acid samples are genomic DNA. The nucleic acid sample need not comprise amplified nucleic acid.

### Amplification of Nucleic Acids

The nucleic acid samples used for a subject are genomic DNA or nucleic acid derived therefrom. The DNA samples of a subject optionally can be fragmented using restriction endonucleases and/or amplified prior to determining GGDS. In preferred embodiments, the DNA fragments are amplified using polymerase chain reaction (PCR). Methods for practicing PCR are well known to those of skill in the art. One advantage of PCR is that small quantities of DNA can be used. For example, genomic DNA from a subject may be about 150 ng, 175, ng, 200 ng, 225 ng, 250 ng, 275 ng, or 300 ng of DNA.

In certain embodiments of the methods of the invention, the nucleic acid from a subject is amplified using a single primer pair. For example, genomic DNA samples can be digested with restriction endonucleases to generate fragments of genomic DNA that are then ligated to an adaptor DNA sequence which the primer pair recognizes (see Example section 6). In other embodiments of the methods of the invention, the nucleic acid of a subject is amplified using sets of primer pairs specific to SNPs loci located throughout the genome. Such sets of primer pairs each recognize genomic DNA sequences flanking a particular SNP. A DNA sample suitable for hybridization can be obtained, e.g., by polymerase chain reaction (PCR) amplification of genomic DNA, fragments of genomic DNA, fragments of genomic DNA ligated to adaptor sequences or cloned sequences. Computer programs that are well known in the art can be used in the design of primers with the desired specificity and optimal amplification properties, such as Oligo version 5.0 (National Biosciences). PCR methods are well known in the art, and are described, for example, in Innis et al., eds., 1990, PCR Protocols: A Guide to Methods And Applications, Academic Press Inc., San Diego, Calif. It will be apparent to one skilled in the art that controlled robotic systems are useful for isolating and amplifying nucleic acids and can be used.

In other embodiments, where genomic DNA of a subject is fragmented using restriction endonucleases and amplified prior to determining GGDS, the amplification can comprise cloning regions of genomic DNA of the subject. In such methods, amplification of the DNA regions is achieved through the cloning process. For example, expression vectors can be engineered to express large quantities of particular fragments of genomic DNA of the subject (Sambrook, J. et al., eds., 1989, Molecular Cloning: A Laboratory Manual, 2nd Ed., Cold Spring Harbor Laboratory Press, Cold Spring Harbor, N.Y., at pp. 9.47-9.51).

In yet other embodiments, where the DNA of a subject is fragmented using restriction endonucleases and amplified prior to determining GGDS, the amplification comprises expressing a nucleic acid encoding a gene, or a gene and flanking genomic regions of nucleic acids, from the subject. RNA (pre-messenger RNA) that comprises the entire transcript including introns is then isolated and used in the methods of the invention to determine GGDS and the phenotype of a cancer.

In certain embodiments, no amplification is required. In such embodiments, the genomic DNA, or pre-RNA, of a subject may be fragmented using restriction endonucleases or other methods. The resulting fragments may be hybridized to SNP probes. Typically, greater quantities of DNA are needed to be isolated in comparison to the quantity of DNA or pre-mRNA needed where fragments are amplified. For example, where the nucleic acid of a subject is not amplified, a DNA sample of a subject for use in hybridization may be about 400 ng, 500 ng, 600 ng, 700 ng, 800 ng, 900 ng, or 1000 ng of DNA or greater.

### Hybridization

The nucleic acid samples derived from a subject used in the methods of the invention can be hybridized to SNP oligonucleotide probes in order to identify informative SNPs in nucleic acid samples from non-cancerous tissues and/or cells of a subject. Hybridization can also be used to determine whether the informative SNPs identified exhibit loss of heterozygosity in nucleic acid samples from cancerous tissues and/or cells of the subject. In preferred embodiments, the SNP oligonucleotide probes used in the methods of the invention comprise an array of probes that can be tiled on a DNA chip. In preferred embodiments, heterozygosity of a SNP locus is determined by a method that does not comprise detecting a change in size of restriction enzyme-digested nucleic acid fragments.

Hybridization and wash conditions used in the methods of the invention are chosen so that the nucleic acid samples from a subject to be analyzed by the invention specifically bind or specifically hybridize to the complementary oligonucleotide sequences of the array, preferably to a specific array site, wherein its complementary DNA is located.

The single-stranded synthetic oligodeoxyribonucleic acid DNA probes of an array may need to be denatured prior to contacting with the nucleic acid samples from a subject, e.g., to remove hairpins or dimers which form due to self complementary sequences.

Optimal hybridization conditions will depend on the length of the probes and type of nucleic acid samples from a subject. General parameters for specific (i.e., stringent) hybridization conditions for nucleic acids are described in Sambrook, J. et al., eds., 1989, Molecular Cloning: A Laboratory Manual, 2nd Ed., Cold Spring Harbor Laboratory Press, Cold Spring Harbor, N.Y., at pp. 9.47-9.51 and 11.55-11.61; Ausubel et al., eds., 1989, Current Protocols in Molecules Biology, Vol. 1, Green Publishing Associates, Inc., John Wiley & Sons, Inc., New York, at pp. 2.10.1-2.10.16. Exemplary useful hybridization conditions are provided in, e.g., Tijessen, 1993, Hybridization With Nucleic Acid Probes, Elsevier Science Publishers B. V. and Kricka, 1992, Nonisotopic DNA Probe Techniques, Academic Press, San Diego, Calif.

Particularly preferred hybridization conditions for use with the screening and/or signaling chips of the present invention include hybridization at a temperature at or near (e.g., within about 5° C.) the mean melting temperature of the probes.

### Oligonucleotide Nucleic Acid Arrays

In the methods of the present invention, DNA arrays can be used to determine whether heterozygosity of a SNP is exhibited in a nucleic acid sample by measuring the level of hybridization of the nucleic acid sequence to oligonucleotide probes that comprise complementary sequences. Hybridization can be used to determine the presence or absence of heterozygosity. Various formats of DNA arrays that employ oligonucleotide “probes,” (i.e., nucleic acid molecules having defined sequences) are well known to those of skill in the art.

Typically, a set of nucleic acid probes, each of which has a defined sequence, is immobilized on a solid support in such a manner that each different probe is immobilized to a predetermined region. In certain embodiments, the set of probes forms an array of positionally-addressable binding (e.g., hybridization) sites on a support. Each of such binding sites comprises a plurality of oligonucleotide molecules of a probe bound to the predetermined region on the support. More specifically, each probe of the array is preferably located at a known, predetermined position on the solid support such that the identity (i.e., the sequence) of each probe can be determined from its position on the array (i.e., on the support or surface). Microarrays can be made in a number of ways, of which several are described herein below. However produced, microarrays share certain characteristics. The arrays are reproducible, allowing multiple copies of a given array to be produced and easily compared with each other.

Preferably, the microarrays are made from materials that are stable under binding (e.g., nucleic acid hybridization) conditions. The microarrays are preferably small, e.g., between about 1 cm² and 25 cm², preferably about 1 to 3 cm². However, both larger and smaller arrays are also contemplated and may be preferable, e.g., for simultaneously evaluating a very large number of different probes.

Oligonucleotide probes can be synthesized directly on a support to form the array. The probes can be attached to a solid support or surface, which may be made, e.g., from glass, plastic (e.g., polypropylene, nylon), polyacrylamide, nitrocellulose, gel, or other porous or nonporous material. The set of immobilized probes or the array of immobilized probes is contacted with a sample containing labeled nucleic acid species so that nucleic acids having sequences complementary to an immobilized probe hybridize or bind to the probe. After separation of, e.g., by washing off, any unbound material, the bound, labeled sequences are detected and measured. The measurement is typically conducted with computer assistance. Using DNA array assays, complex mixtures of labeled nucleic acids, e.g., nucleic acid fragments derived a restriction digestion of genomic DNA from non-cancerous tissue, can be analyzed. DNA array technologies have made it possible to determine heterozygosity of a large number of SNPs at different loci throughout the genome.

In certain embodiments, high-density oligonucleotide arrays are used in the methods of the invention. These arrays containing thousands of oligonucleotides complementary to defined sequences, at defined locations on a surface can be synthesized in situ on the surface by, for example, photolithographic techniques (see, e.g., Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; 5,510,270; 5,445,934; 5,744,305; and 6,040,138). Methods for generating arrays using inkjet technology for in situ oligonucleotide synthesis are also known in the art (see, e.g., Blanchard, International Patent Publication WO 98/41531, published Sep. 24, 1998; Blanchard et al., 1996, Biosensors And Bioelectronics 11:687-690; Blanchard, 1998, in Synthetic DNA Arrays in Genetic Engineering, Vol. 20, J. K. Setlow, Ed., Plenum Press, New York at pages 111-123). Another method for attaching the nucleic acids to a surface is by printing on glass plates, as is described generally by Schena et al. (1995, Science 270:467-470). Other methods for making microarrays, e.g., by masking (Maskos and Southern, 1992, Nucl. Acids. Res. 20:1679-1684), may also be used. When these methods are used, oligonucleotides (e.g., 15 to 60-mers) of known sequence are synthesized directly on a surface such as a derivatized glass slide. The array produced can be redundant, with several oligonucleotide molecules corresponding to each SNP locus.

One exemplary means for generating the oligonucleotide probes of the DNA array is by synthesis of synthetic polynucleotides or oligonucleotides, e.g., using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 15 and about 600 bases in length, more typically between about 20 and about 100 bases, most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., 1993, Nature 363:566-568; U.S. Pat. No. 5,539,083). In alternative embodiments, the hybridization sites (i.e., the probes) are made from plasmid or phage clones of regions of genomic DNA corresponding to SNPs or the complement thereof.

The size of the SNP oligonucleotide probes used in the methods of the invention preferably is at least 10, 20, 25, 30, 35, 40, 45, or 50 nucleotides in length. In preferred embodiments of the invention, probes of 25 nucleotides are used. It is well known in the art that although hybridization is selective for complementary sequences, other sequences which are not perfectly complementary may also hybridize to a given probe at some level. Thus, multiple oligonucleotide probes with slight variations can be used, to optimize hybridization of samples. To further optimize hybridization, hybridization stringency condition, e.g., the hybridization temperature and the salt concentrations, may be altered by methods that are well known in the art.

In preferred embodiments, the high-density oligonucleotide arrays used in the methods of the invention comprise oligonucleotides corresponding to SNPs. The oligonucleotide probes may comprise DNA or DNA “mimics” (e.g., derivatives and analogues) corresponding to a portion of each SNP locus in a subject's genome. The oligonucleotide probes can be modified at the base moiety, at the sugar moiety, or at the phosphate backbone. Exemplary DNA mimics include, e.g., phosphorothioates. For each SNP locus, a plurality of different oligonucleotides may be used that are complementary to the sequences of sample nucleic acids. For example, for a single SNP about 2, 3, 4, 5, 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more different oligonucleotides can be used. Each of the oligonucleotides for a particular SNP may have a slight variation in perfect matches, mismatches, and flanking sequence around the SNP. In certain embodiments, the SNP probes are generated such that the probes for a particular SNP comprise overlapping and/or successive overlapping sequences which span or are tiled across a genomic region containing the SNP site, where all the probes contain the SNP site. By way of example, overlapping probe sequences can be tiled at steps of a predetermined base intervals, e. g. at steps of 1, 2, 3, 4, 5, 6, 7, 8, 9, or 10 bases intervals.

In certain embodiments, the heterozygosity of SNPs is determined using pairs of SNP probes for each heterozygous SNP of (a), where the pair of SNP probes for each SNPs correspond to a match and a mismatch, respectively, at the polymorphic nucleotide of the SNP site.

For oligonucleotide probes targeted at nucleic acid species of closely resembled (i.e., homologous) sequences, “cross-hybridization” among similar probes can significantly contaminate and confuse the results of hybridization measurements. Cross-hybridization is a particularly significant concern in the detection of SNPs since the sequence to be detected (i.e., the particular SNP) must be distinguished from other sequences that differ by only a single nucleotide. Cross-hybridization can be minimized by regulating either the hybridization stringency condition and/or during post-hybridization washings. Highly stringent conditions allow detection of allelic variants of a nucleotide sequence, e.g., about 1 mismatch per 10-30 nucleotides.

There is no single hybridization or washing condition which is optimal for all different nucleic acid sequences. For particular arrays of SNPs, these conditions can be identical to those suggested by the manufacturer or can be adjusted by one of skill in the art.

In preferred embodiments, the SNP oligonucleotide probes used in the methods of the invention are immobilized (i.e., tiled) on a glass slide called a chip. For example, a DNA microarray can comprises a chip on which oligonucleotides (purified single-stranded DNA sequences in solution) have been robotically printed in an (approximately) rectangular array with each spot on the array corresponds to a single DNA sample which encodes an oligonucleotide. In summary the process comprises, flooding the DNA microarray chip with a labeled sample under conditions suitable for hybridization to occur between the slide sequences and the labeled sample, then the array is washed and dried, and the array is scanned with a laser microscope to detect hybridization. In certain embodiments there are about 5,000 to 7,000, 6,000 to 8,000, 7,000 to 9,000, 8,000 to 10,000, 9,000 to 11,000, 10,000 to 12,000, 11,000 to 13,000, 12,000 to 14,000, 13,000 to 15,000, 14,000 to 16,000, 15,000 to 17,000, 16,000 to 18,000, 17,000 to 19,000, 18,000 to 20,000 or more SNPs for which probes appear on the array (with match/mismatch probes for a single SNP or probes tiled across a single SNP site counting as one SNP). The maximum number of SNPs being probed per array is determined by the size of the genome and genetic diversity of the subjects species. DNA chips are well known in the art and can be purchased in pre-fabricated form with sequences specific to particular species. In a preferred embodiment, the GeneChip™ HuSNP Mapping 10K array (Affymetrix, Santa Clara, Calif.) is used in the methods of the invention.

### Signal Detection

In preferred embodiments, nucleic acid samples derived from a subject are hybridized to the binding sites of the array (e.g., SNP oligonucleotide chip). In certain embodiments, nucleic acid samples derived from each of the two sample types of a subject (i.e., cancerous and non-cancerous) are hybridized to separate, though identical, SNP oligonucleotide chips. In certain embodiments, nucleic acid samples derived from one of the two sample types of a subject (i.e., cancerous and non-cancerous) is hybridized to a SNP oligonucleotide chip, then following signal detection the chip is washed to remove the first labeled sample and reused to hybridize the remaining sample. Preferably the chip is not reused more than once. In certain embodiments, the nucleic acid samples derived from each of the two sample types of a subject (i.e., cancerous and non-cancerous) are differently labeled so that they can be distinguished. When the two samples are mixed and hybridized to the same array, the relative intensity of signal from each sample is determined for each site on the array, and any relative difference in abundance of an allele of a SNP locus detected.

Signals can be recorded and, in a preferred embodiment, analyzed by computer, e.g., using a 12 bit or 16 bit analog to digital board (see Section 5.79). In one embodiment, the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the array, a ratio of the emission of the two fluorophores can be calculated, which may help in eliminating cross hybridization signals to more accurately determining whether a particular SNP locus is heterozygous or homozygous.

### Labeling

In preferred embodiments, the nucleic acids samples, fragments thereof, or fragments thereof ligated to adaptor regions used in the methods of the invention are detectably labeled.

In certain embodiments of the methods of the invention, the detectable label is a fluorescent label, e.g., by incorporation of nucleotide analogues. Other labels suitable for use in the present invention include, but are not limited to, biotin, iminobiotin, antigens, cofactors, dinitrophenol, lipoic acid, olefinic compounds, detectable polypeptides, electron rich molecules, enzymes capable of generating a detectable signal by action upon a substrate, and radioactive isotopes.

Radioactive isotopes include that can be used in conjunction with the methods of the invention, but are not limited to, ³²p and ¹⁴C. Fluorescent molecules suitable for the present invention include, but are not limited to, fluorescein and its derivatives, rhodamine and its derivatives, texas red, 5′carboxy-fluorescein (“FAM”), 2′, 7′-dimethoxy-4′, 5′-dichloro-6-carboxy-fluorescein (“JOE”), N, N, N′, N′-tetramethyl-6-carboxy-rhodamine (“TAMRA”), 6-carboxy-X-rhdoamine (“ROX”), HEX, TET, IRD40, and IRD41.

Fluorescent molecules which are suitable for use according to the invention further include: cyamine dyes, including but not limited to Cy2, Cy3, Cy3.5, CY5, Cy5.5, Cy7 and FLUORX; BODIPY dyes including but not limited to BODIPY-FL, BODIPY-TR, BODIPY-TMR, BODIPY-630/650, and BODIPY-650/670; and ALEXA dyes, including but not limited to ALEXA-488, ALEXA-532, ALEXA-546, ALEXA-568, and ALEXA-594; as well as other fluorescent dyes which will be known to those who are skilled in the art. Electron rich indicator molecules suitable for the present invention include, but are not limited to, ferritin, hemocyanin, and colloidal gold.

Two-color fluorescence labeling and detection schemes may also be used (Shena et al., 1995, Science 270:467-470). Use of two or more labels can be useful in detecting variations due to minor differences in experimental conditions (e.g., hybridization conditions). In some embodiments of the invention, at least 5, 10, 20, or 100 dyes of different colors can be used for labeling. Such labeling would also permit analysis of multiple samples simultaneously which is encompassed by the invention.

The labeled nucleic acid samples, fragments thereof, or fragments thereof ligated to adaptor regions that can be used in the methods of the invention are contacted to a plurality of oligonucleotide probes under conditions that allow sample nucleic acids having sequences complementary to the probes to hybridize thereto.

Depending on the type of label used, the hybridization signals can be detected using methods well known to those of skill in the art including, but not limited to, X-Ray film, phosphor imager, or CCD camera. When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Res. 6:639-645). In a preferred embodiment, the arrays are scanned with a laser fluorescence scanner with a computer controlled X-Y stage and a microscope objective. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al., 1996, Genome Res. 6:639-645. Alternatively, a fiber-optic bundle can be used such as that described by Ferguson et al., 1996, Nature Biotech. 14:1681-1684. The resulting signals can then be analyzed to determine the presence or absence of heterozygosity or homozygosity for informative SNPs using computer software as described below in Section 5.14.

### Wave™ Hybridization Analysis

In one embodiment, as an alternative or additionally to more standard hybridization methods, SNP heterozygosity or absence thereof is detected using the WAVE™ nucleic acid fragment analysis system (Tansgenomic, Inc. Omaha, Nebr.). First, an analysis of PCR product size, yield, and purity is carried out in a non-denaturing manner at 50° C. The results of the analysis are plotted as absorbance (mV) versus retention time (min), where the height of peaks in the graph correlate to size of PCR fragments. Second, denaturing high performance liquid chromatography (DHPLC) is used to detect unknown DNA sequence variants by comparing to a reference sample, i.e., non-cancerous genomic DNA. Detection of SNPs, insertions and deletions are based on the formation of heteroduplexes of the non-cancerous and cancerous amplicons. Under denaturing conditions, the heteroduplexes elute earlier than the homoduplexes. Software is used to predict the optimal temperature for DHPLC analysis. Heteroduplex peaks can be rapidly identified in the resulting chromatogram, which indicate the presence of SNPs insertions, and deletions. Elution profiles that differ from the non-cancerous or cancerous DNA indicate the presence of mutations or polymorphisms.

### Algorithms for Determining Heterozygosity

Once the hybridization signal has been detected the resulting data can be analyzed using algorithms. In certain embodiments, the algorithm for determining heterozygosity at a SNP locus is based on identifying the number of informative SNPs that remain heterozygous in a nucleic acid sample from cancerous tissue and/or cells of a subject. In other embodiments, the algorithm for determining heterozygosity at a SNP is based on identifying the number of informative SNPs that have lost heterozygosity in a nucleic acid sample from cancerous tissue and/or cells of a subject.

In one embodiment, the algorithm for determining heterozygosity is based on identifying a locus as having allele loss (ie., absence of heterozygosity) if it is heterozygous in the noncancerous sample(s) and if the change in relative allele score (RAS) in the cancerous sample is >0.5 regardless of the allele call in the cancerous. Change in RAS is the difference in the relative allele signal intensities between noncancerous and cancerous specimens.

In one embodiment, the algorithm for determining heterozygosity is based on identifying a locus as having allele loss if it is heterozygous in the noncancerous sample(s) and if the change in RAS in the cancerous sample is >0.4.

In a preferred embodiment, a locus is determined to have allele loss if it is heterozygous in the noncancerous sample(s) and if the change in RAS in the cancerous sample is >0.354, which is equivalent to a signal intensity reduction of 50% on a traditional gel analysis.

In one embodiment, the algorithm for determining heterozygosity is based on identifying a locus as having allele loss if it is heterozygous in the noncancerous sample(s) and if the change in RAS in the noncancerous sample is >0.3.

In one embodiment, the algorithm for determining heterozygosity is based on identifying a locus as having allele loss if it is heterozygous in the noncancerous sample(s) and if the change in RAS in the noncancerous sample is >0.2.

In one embodiment, the algorithm for determining heterozygosity is based on identifying a locus as having allele loss if it is heterozygous in the noncancerous sample(s), and if the change in RAS in the noncancerous sample is >0.5.

In one embodiment, the algorithm for determining heterozygosity is based on identifying a locus as having allele loss if it is heterozygous in the noncancerous sample(s), and if the change in RAS in the noncancerous sample is >0.4.

In one embodiment, the algorithm for determining heterozygosity is based on identifying a locus as having allele loss if it is heterozygous in the noncancerous sample(s), and if the change in RAS in the noncancerous sample is >0.354 which is equivalent to a signal intensity reduction of 50% on a traditional gel analysis.

In one embodiment, the algorithm for determining heterozygosity is based on identifying a locus as having allele loss if it is heterozygous in the noncancerous sample(s), and if the change in RAS in the noncancerous sample is >0.3.

In one embodiment, the algorithm for determining heterozygosity is based on identifying a locus as having allele loss if it is heterozygous in the noncancerous sample(s), and if the change in RAS in the noncancerous sample is >0.2.

In certain preferred embodiments, the above described algorithms can be used to determine heterozygosity or homozygosity of the informative SNPs using computer programs, such as those described below in Section 5.14.

### Computer Implementation Systems and Methods

In certain preferred embodiments, the methods of the invention are implemented using a computer program. For example, a computer program can be used to compare the number of (informative) heterozygous SNPs identified from the non-cancerous sample(s) (i.e., value of (a)) to either the number of loci having retention of heterozygosity or the number of loci having loss of heterozygosity of those same informative loci (i.e., value of (b)) in nucleic acid samples derived from the cancerous sample of the subject, e.g., to compute the desired ratio or logarithm thereof.

The methods of the present invention can preferably be implemented using a computer system, such as the computer system described in this section, according to the following programs and methods to analyze SNP hybridization signals and optionally calculate a GGDS for a subject that is determinative and/or predictive of the phenotype of a cancer in the subject. A computer system can also preferably store and manipulate data generated by the methods of the present invention which comprises a plurality of hybridization signal changes/profiles during approach to equilibrium in different hybridization measurements and which can be used by a computer system in implementing the methods of this invention. In certain embodiments, a computer system receives SNP probe hybridization data; (ii) stores SNP probe hybridization data; and (iii) compares SNP probe hybridization data to determine whether an absence or presence of SNP heterozygosity has occurred in said nucleic acid sample from cancerous or precancerous tissue. In certain embodiments, the comparison is carried out using the algorithms described in Section 5.13. In certain embodiments, the GGDS is calculated. In certain embodiments, a computer system (i) compares the determined GGDS to a threshold value; and (ii) outputs an indication of whether said GGDS is above or below a threshold value, or a phenotype based on said indication. In certain embodiments, such computer systems are also considered part of the present invention.

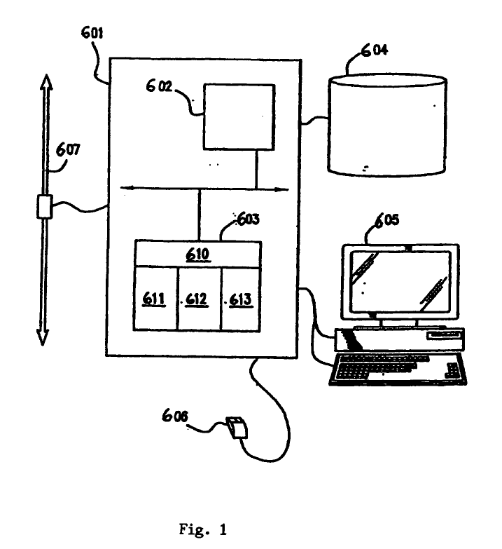

Numerous types of computer systems can be used to implement the analytic methods of this invention an example of a computer system that can be used is illustrated in FIG. 1. An exemplary computer system suitable from implementing the methods of this invention can be an Intel PENTIUM T-BASED processor of 200 MHZ or greater clock rate and with 32 MB or more main memory. In a preferred embodiment, computer system 601 is a cluster of a plurality of computers comprising a head “node” and eight sibling “nodes,” with each node having a central processing unit (“CPU”). In addition, the cluster also comprises at least 128 MB of random access memory (“RAM”) on the head node and at least 256 MB of RAM on each of the eight sibling nodes. Therefore, the computer systems of the present invention are not limited to those consisting of a single memory unit or a single processor unit. The external components can include a mass storage 604. This mass storage can be one or more hard disks that are typically packaged together with the processor and memory. Such hard disk are typically of 1 GB or greater storage capacity and more preferably have at least 6 GB of storage capacity. For example, in a preferred embodiment, described above, wherein a computer system of the invention comprises several nodes, each node can have its own hard drive. The head node preferably has a hard drive with at least 6 GB of storage capacity whereas each sibling node preferably has a hard drive with at least 9 GB of storage capacity. A computer system of the invention can further comprise other mass storage units including, for example, one or more floppy drives, one more CD-ROM drives, one or more DVD drives or one or more DAT drives.

Other external components typically include a user interface device 605, which is most typically a monitor and a keyboard together with a graphical input device 606 such as a “mouse.” The computer system is also typically linked to a network link 607 which can be, e.g., part of a local area network (“LAN”) to other, local computer systems and/or part of a wide area network (“WAN”), such as the Internet, that is connected to other, remote computer systems. For example, in the preferred embodiment, discussed above, wherein the computer system comprises a plurality of nodes, each node is preferably connected to a network, preferably an NFS network, so that the nodes of the computer system communicate with each other and, optionally, with other computer systems by means of the network and can thereby share data and processing tasks with one another.

Several software components can be loaded into memory during operation of such a computer system. The software components can comprise both software components that are standard in the art and components that are special to the present invention. These software components are typically stored on mass storage such as the hard drive 604, but can be stored on other computer readable media as well including, for example, one or more floppy disks, one or more CD-ROMs, one or more DVDs or one or more DATs. Software component 610 represents an operating system which is responsible for managing the computer system and its network interconnections. The operating system can be, for example, of the Microsoft Windows family such as Windows 95, Window 98, Windows NT or Windows 2000. Alternatively, the operating software can be a Macintosh operating system, a UNIX operating system or the LINUX operating system. Software components 611 comprises common languages and functions that are preferably present in the system to assist programs implementing methods specific to the present invention. Languages that can be used to program the analytic methods of the invention include, for example, C and C++, FORTRAN, PERL, HTML, JAVA, and any of the UNIX or LINUX shell command languages such as C shell script language. The methods of the invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

Software component 612 comprises analytic methods of the present invention, preferably programmed in a procedural language or symbolic package. For example, software component 612 preferably includes programs that cause the processor to implement steps of accepting a plurality of hybridization signals (i.e., signal profiles of a sample) and storing the profiles data in the memory. For example, the computer system can accept hybridization signal profiles that are manually entered by a user (e.g., by means of the user interface). More preferably, however, the programs cause the computer system to retrieve hybridization signal profiles from a storage medium or a database. Such a database can be stored on a mass storage (e.g., a hard drive) or other computer readable medium and loaded into the memory of the computer, or the compendium can be accessed by the computer system by means of the network 607.

In an exemplary implementation to practice the methods of the present invention, hybridization data (e.g., one or more measured hybridization levels or curves, etc.) (613) contained in a database and/or loaded into the memory of the computer system is represented by a data structure comprising a plurality of data fields.

In particular, the data structure for a particular hybridization signal profile will comprise a separate data field for each time at which a measured value, e.g., hybridization level, is an element of the hybridization signal profile. The analytic software component 612 comprises programs and/or subroutines which can cause the processor to perform steps of comparing said hybridization level measured at a first time to the hybridization level measured at a second time or the measured hybridization levels of more than one time in said hybridization signal profile, for each of said plurality of hybridization signal profiles (e.g., signal profiles of hybridization of samples derived from cancerous and noncancerous tissue). The computer then output and display the calculated differences, including but are not limited to arithmetic difference, ratio, etc., in the measured hybridization levels for each first and second time as a measure of the rate of hybridization signal changes between said first and second time.

In certain embodiments, the invention provides for a computer comprising: a central processing unit; a memory, coupled to the central processing unit, the memory storing: (i) instructions for computing a GGDS for cancerous or precancerous tissue, wherein said GGDS is a relative measure of (a) number of heterozygous SNPs in a plurality of heterozygous SNPs, said plurality of heterozygous SNPs consisting of different SNPs wherein heterozygosity occurs in genomic DNA of non-cancerous tissue of said species to which said subject belongs, wherein said number of heterozygous SNPs in said plurality is in excess of 100 SNPs; and (b) the number of SNPs for which heterozygosity is determined to be present, or the number of SNPs for which heterozygosity is determined to be absent, among the number of heterozygous SNPs in said plurality of (a), in a nucleic acid sample of, or derived from, genomic DNA of cancerous or precancerous tissue of the subject. In certain embodiments, the memory further stores: (ii) instructions for comparing said GGDS to a threshold value; and (iii) instructions for outputing an indication of whether said GGDS is above or below a threshold value, or a phenotype based on said indication. In certain embodiments, the memory further stores in a database said number of heterozygous SNPs of (a). In certain embodiments, the memory further stores in a database an indication of the identity (e.g., sequence, and/or genetic locus (location), and/or a location on an array which correlates to a locus) of each SNP in the heterozygous SNPs of (a). In certain embodiments, the number of heterozygous SNPs of (a) comprises heterozygous SNPs from noncancerous tissue of a plurality of members of said species, and wherein said identity of each heterozygous SNP in the database is associated with an identifier for which organism exhibits said heterozygous SNP. In certain embodiments, the memory further stores: (i) instructions for receiving SNP probe hybridization data; (ii) instructions for storing SNP probe hybridization data; (iii) instructions for comparing SNP probe hybridization data to determine whether an absence or presence of SNP heterozygosity has occurred in said nucleic acid sample from cancerous or precancerous tissue.

In certain embodiments, the computer comprises a database for storage of hybridization signal profiles. Such stored profiles can be accessed and used to calculate GGDS. For example, of the hybridization signal profile of a sample derived from the noncancerous tissue of a subject were stored, it could then be compared to the hybridization signal profile of a sample derived from the cancerous tissue of the subject. Preferably, such a database will be in an electronic form that can be loaded into a computer system 601. Such electronic forms include databases loaded into the main memory 603 of a computer system used to implement the methods of this invention, or in the main memory of other computers linked by network connection 607, or embedded or encoded on mass storage media 604, or on removable storage media such as a DVD-ROM, CD-ROM or floppy disk. In related embodiments, the computer further comprises a database for storing the value of (a). In certain embodiments, the computer contains a computer program mechanism comprising instructions for software can be used to compute GGDS based on the SNP hybridization signal output and compare to GGDS threshold values for a phenotype (e.g. threshold values described below in Sections 5.2, 5.3, 5.4, 5.5, and 5.8.1) to determine and/or predict the phenotype of a cancer and output the predicted phenotype.

According to certain aspects of the invention, a computer program product is provided, for use in conjunction with a computer system, the computer program product comprising a computer readable storage medium and a computer program mechanism embedded therein, the computer program mechanism comprising: (i) instructions for computing a GGDS for cancerous or precancerous tissue, wherein said GGDS is a relative measure of (a) number of heterozygous SNPs in a plurality of heterozygous SNPs, said plurality of heterozygous SNPs consisting of different SNPs wherein heterozygosity occurs in genomic DNA of non-cancerous tissue of said species to which said subject belongs, wherein said number of heterozygous SNPs in said plurality is in excess of 100 SNPs; and (b) the number of SNPs for which heterozygosity is determined to be present, or the number of SNPs for which heterozygosity is determined to be absent, among the number of heterozygous SNPs in said plurality of (a), in a nucleic acid sample of, or derived from, genomic DNA of cancerous or precancerous tissue of the subject. In certain embodiments, the computer program mechanism further comprises: (ii) instructions for comparing said GGDS to a threshold value; and (iii) instructions for outputing an indication of whether said GGDS is above or below a threshold value, or a phenotype based on said indication. In certain embodiments, the memory further stores in a database said number of heterozygous SNPs of (a). In certain embodiments, the memory further stores in a database an indication of the identity of each SNP in the heterozygous SNPs of (a). In certain embodiments, the number of heterozygous SNPs of (a) comprises heterozygous SNPs from noncancerous tissue of a plurality of members of said species, and wherein said identity of each heterozygous SNP in the database is associated with an identifier for which organism exhibits said heterozygous SNP. In certain embodiments, the memory further stores: (i) instructions for receiving SNP probe hybridization data; (ii) instructions for storing SNP probe hybridization data; (iii) instructions for comparing SNP probe hybridization data to determine whether an absence or presence of SNP heterozygosity has occurred in said nucleic acid sample from cancerous or precancerous tissue. In certain embodiments, the computer program product is stored, for example, on a DVD-ROM, CD-ROM or floppy disk. The computer program product can be packaged with means for hybridization to probes for the heterozygous SNPs, in a kit.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

### Kits of the Invention

The present invention provides kits for practicing the methods of the present invention. In certain embodiment, the invention provides a kit comprising (a) nucleic acid probes comprising SNP hybridization probes, said SNP hybridization probes comprising nucleotide sequences complementary to a plurality of SNPs, respectively, said SNPs consisting of at least 100 different SNPs wherein heterozygosity occurs in genomic DNA of non-cancerous tissue of the same species; and (b) a computer program product for use in conjunction with a computer system, the computer program product comprising a computer readable storage medium and a computer program mechanism embedded therein, the computer program mechanism comprising instructions for determining a relative measure of (i) the number of at least 100 different SNPs in (a), and (ii) the number of SNPs for which heterozygosity is determined to be present, or the number of SNPs for which heterozygosity is determined to be absent, among the at least 100 different SNPs of (a) in a nucleic acid sample of, or derived from, genomic DNA of cancerous tissue of a subject of said species.

In certain embodiments, the nucleic acid probes are attached to a solid or semi-solid phase. By way of example, the kit may also comprise a device or a component of a device for performing the methods of the invention, for example a SNP oligonucleotide chip. The kit may also comprise 100 or more of the SNP probes or pairs of probes described above. The kit may also comprise a computer and/or computer program products (e.g., a CD-ROM, floppy disk, or DVD) for determining GGDS as described in Section 5.14.

### EXAMPLE 1

### Determining GGDS in Lung Tumor Samples

### Introduction

The Example presented herein describes determining GGDS in non-small-cell lung cancer patients and the successful prognosis of the clinical outcome of cancer based on this determination.

A genome-wide genotyping method was used to successfully determine global genome damage to DNA in individual cancer samples; the quantification of the extent of such damage significantly correlated to clinical outcome of the cancer.

In contrast to the prior art described in the background section above, the SNP array analysis according to the present invention provides for use of a greater number of informative loci and a genome-wide distribution of informative loci for use in allele loss analysis as an indicator of global genome damage.

### Materials and Methods

Determining Loss Of Heterozygosity. To assess whether global genome damage impacts the clinical outcome of cancer, a genome-wide high throughout genotyping method was used. The method was based on match/mismatch hybridization of amplified genomic DNA to SNP-specific oligonucleotides spotted on glass slides for global genome damage assessment (GeneChip™ HuSNP Mapping 10K array, Affymetrix, Santa Clara, Calif.) (see Data Sheet for GeneChip™ Human Mapping 10K array and Assay Kit, 2003 available at the Affymetrix website). SNPs are the most abundant DNA markers with an estimated frequency of 1 SNP in every 1000 bases. There are six possible SNP types, either transitions (A<>T or G<>C) or transversions (A<>G, A<>C, G<>T or C<>T). The 11,560 SNPs on the array had been selected based on genomic distribution, Hardy-Weinberg equilibrium, and informativeness (median heterozygosity 36%, 25th percentile 22% and 75th percentile 47%). The median distance between the SNPs on the array was about 150 kb and the average distance between SNPs was 210 kb. For each SNP, 40 different 25 bp oligonucleotides were tiled on the DNA chip. Each of the 40 oligonucleotides for a SNP had a slight variation in perfect matches, mismatches, and flanking sequence around the SNP. The DNA chip comprised more than 1 million copies of each of the 25 bp oligonucleotides. A total of 250 ng DNA was required to obtain reliable signals. The method had an average genotype reproducibility of 99.65% when compared to standard techniques.

Primary lung tumor samples were collected and matched with noncancerous lung tissue samples from 44 patients that had undergone complete surgical resection for non-small-cell lung cancer (NSCLC). None of these patients had received radiation or chemotherapy before surgical resection. Demographic, epidemiologic, clinical, and follow-up information on each of these patients had been recorded following Institutional Review Board approved protocols. All specimens had been reviewed to confirm tissue diagnosis and were microdissected to reduce the amount of normal tissue contamination. Genomic DNA was extracted from isolated cancerous tissue and tissue that appeared to be noncancerous, i.e., normal tissue. The DNA samples were quantified and assessed for integrity by standard techniques. DNA amplification and array hybridization were performed as specified by the manufacturer. Briefly, each 250 ng DNA was digested with the restriction enzyme XbaI to produce fragments of varying size. An adapter that recognizes cohesive four base pair overhangs was then ligated to the ends of each fragment. A single primer that recognized the adapter sequence was used with PCR to amplify the adapter ligated DNA fragments. The PCR conditions were optimized to amplify fragments that were about 250 to 1,000 bp in size. The amplification product was then fragmented, labeled and hybridized to the GeneChip™ HuSNP Mapping 10K array.

Hybridization signals were captured with a GCS 3000 scanner (Affymetrix, Santa Clara, Calif.), and data were analyzed using GeneChip DNA analysis software, version 2.0 (Affymetrix, Santa Clara, Calif.) to identify heterozygous loci in normal tissue samples.

For each of the heterozygous loci identified in normal DNA from the 44 patients, the allele signal in the corresponding tumor DNA was analyzed with 11 different algorithms to determine whether or not allele loss was present or absent. The first algorithm for determining heterozygosity was based on identifying a locus as having allele loss if it was heterozygous in the normal sample and homozygous in the cancerous sample. The second algorithm for determining heterozygosity was based on identifying a locus as having allele loss if it was heterozygous in the normal sample and if the change in Relative Allele Signal (RAS) in the tumor sample was >0.5 regardless of the allele call in the tumor and the change in RAS was the difference in the relative allele signal intensities between normal and tumor specimens. The RAS score was determined as follows: if the allele call was A then the RAS was scored as 1, if the allele call was B then the RAS was scored as 0, and if the allele call was AB the RAS was scored as 0.5. The third algorithm for determining heterozygosity was based on identifying a locus as having allele loss if it was heterozygous in the normal sample and if the change in RAS in the tumor sample was >0.4. The fourth algorithm used for determining heterozygosity was based on identifying a locus as having allele loss if it was heterozygous in the normal sample and if the change in RAS in the tumor sample was >0.354, which was equivalent to a signal intensity reduction of 50% on a traditional gel analysis. The fifth algorithm used for determining heterozygosity was based on identifying a locus as having allele loss if it was heterozygous in the normal sample and if the change in RAS in the tumor sample was >0.3. The sixth algorithm used for determining heterozygosity was based on identifying a locus as having allele loss if it was heterozygous in the normal sample and if the change in RAS in the tumor sample was >0.2. The seventh algorithm used for determining heterozygosity was based on identifying a locus as having allele loss if it was heterozygous in the normal sample and the tumor sample, and if the change in RAS in the tumor sample was >0.5. The eighth algorithm used for determining heterozygosity was based on identifying a locus as having allele loss if it was heterozygous in the normal sample and the tumor sample, and if the change in RAS in the tumor sample was >0.4. The ninth algorithm used for determining heterozygosity was based on identifying a locus as having allele loss if it was heterozygous in the normal sample and the tumor sample, and if the change in RAS in the tumor sample was >0.354 which was equivalent to a signal intensity reduction of 50% on a traditional gel analysis. The tenth algorithm used for algorithm for determining heterozygosity was based on identifying a locus as having allele loss if it was heterozygous in the normal sample and the tumor sample, and if the change in RAS in the tumor sample was >0.3. The eleventh algorithm used for determining heterozygosity was based on identifying a locus as having allele loss if it was heterozygous in the normal sample and the tumor sample, and if the change in RAS in the tumor sample was >0.2. The fourth algorithm was used for subsequent investigations, since it was approximately equivalent to a 50% reduction in allele signal intensities in traditional gel analyses.

For each of the 44 patients, a global genome damage score (GGDS) was then calculated by dividing the number of loci with evidence for loss of heterozygosity by the total number of informative loci. For each of the eleven algorithms, the GGDS values calculated were analyzed for the patient population using standard statistical methods to determine the median, mean, standard deviation, and range limits of the GGDS values for the patient population. The degree of statistical correlation among the statistical GGDS population values calculated using each algorithm was determined by calculating the Spearman correlation coefficient.

Correlation To Clinical Data. GGDS population values were also calculated for subpopulations of patients categorized based on gender, age, smoking status, histopathology, cancer stage, Eastern Cooperative Ontology Group-Performance Status (ECOG-PS) score, and weight loss. The categories were further subdivided. Gender was divided into women and men. Smoking was divided into active smokers who were patients that had not quit smoking or claimed to have quit for less than 1 year prior to diagnosis, former smokers who had quit for more than one year, and never smokers who's life-time consumption of cigarettes was less than 100. Histopathology was divided into squamous and non-squamous. Cancer stage was divided as follows, stage III/IV encompassed 5 patients with stage IIA, 2 with IIIB (Both had T4 disease as a result of a 2nd tumor nodule in the same lobe of the lung as the primary lung cancer; one had N0 and the other N1 lymph node involvement), and 2 with stage IV disease (both had stage IV disease as result of a 2nd tumor nodule in a different lobe of the lung as the primary cancer; both had no evidence for lymph node involvement or other distant metastatic disease). ECOG-PS was further divided based on a value of zero or greater than zero. Weight loss was divided into absent, present, and unknown. The age category was analyzed by calculating the median and range of ages. The GGDS values for these subpopulations were then analyzed using standard statistical methodology to determine the GGDS median values, GGDS range values, and GGDS p-values for each subpopulation.

To determine whether GGDS would be predictive of overall and disease-free survival (OS and DFS) of the 44 patients with completely resected NSCLC, Kaplan-Meier survival curves were plotted by GGDS value, where the x-axis was time in months and the y-axis was either percent OS or DFS. Kaplan-Meier survival curves estimate the survival for long-term periods, based on data from shorter clinical trials. OS was measured from the date of diagnosis to the date of death and DFS was measured from the date of surgery to the date of disease recurrence. The cohort was dichotomized into high versus low GGDS based on the cohort median (0.049). The GGDS patient population data was divided into two categories based on GGDS scores with the first category consisting of GGDS values greater than 0.049 (N=22) and the second less than 0.049 (N=22). Kaplan-Meier survival curves were plotted for each GGDS category for both OS and DFS.

The GGDS patient population data was also analyzed by dividing patients into four categories based on GGDS scores with the first category consisting of GGDS values less than 0.022 (N=11), the second with GGDS values between 0.022 and 0.049 (N=11), the third with GGDS values between 0.049 and 0.090 (N=11), and the forth with GGDS values greater than 0.090 (N=11). Kaplan-Meier survival curves were plotted for the four GGDS categories for OS.

Looking at all possible cut points for cohort dichotomization and keeping group sizes above ten, the optimal cut point for OS was achieved using a GGDS of 0.041. The GGDS patient population data was divided into two categories based on GGDS scores with the first category consisting of GGDS values greater than 0.041 (N=28), and the second less than 0.041 (N=16). Kaplan-Meier survival curves were plotted for each GGDS category for OS.

### Results

Determining Loss Of Heterozygosity. In the 44 DNA samples from normal tissue, the median call rate for all markers on the chip was 93.65% (range 78.09%-98.09%). The median number of heterozygous SNPs was 3,652 or about 33.4% (range 1,8864,033; 20.9-35.8%). This was equivalent to one heterozygous SNP locus every 821,000 bp (range 744,000-1,591,000 bp) on the entire human genome.

As shown in Table 1, the GGDS values for the patient population calculated using the eleven algorithms were highly correlated having a Spearman correlation coefficient of p<0.0001. Using the fourth algorithm, the GGDS ranged from 0.003 to 0.204 with a median of 0.049 indicating that between 0.3% to 20.4% of the entire genome was damaged in lung tumors.

TABLE 1

| Variable | Minimum | Maximum | Median | Mean | Std Dev |
|---|---|---|---|---|---|
| GGDS 1 | 0.00081 | 0.17992 | 0.02335 | 0.04213 | 0.04709 |
| GGDS 2 | 0.00192 | 0.12671 | 0.02038 | 0.02392 | 0.02467 |
| GGDS 3 | 0.00274 | 0.16189 | 0.03472 | 0.04422 | 0.03968 |
| GGDS 4* | 0.00302 | 0.20425 | 0.04930 | 0.06457 | 0.05356 |
| GGDS 5 | 0.00521 | 0.30222 | 0.09419 | 0.10629 | 0.07898 |
| GGDS 6 | 0.02714 | 0.48983 | 0.25206 | 0.25662 | 0.13289 |
| GGDS 7 | 0 | 0.05187 | 0.00401 | 0.00778 | 0.01172 |
| GGDS 8 | 0.00027 | 0.09418 | 0.00948 | 0.01824 | 0.02353 |
| GGDS 9 | 0.00054 | 0.11874 | 0.01697 | 0.02663 | 0.03126 |
| GGDS 10 | 0.00191 | 0.17106 | 0.03841 | 0.04593 | 0.04476 |
| GGDS 11 | 0.02140 | 0.33303 | 0.14727 | 0.14946 | 0.08132 |

Correlation To Clinical Data. Table 2 summarizes the GGDS population values calculated for subpopulations of patients categorized based on gender, age, smoking status, histopathology, cancer stage, ECOG-PS score, and weight loss.

TABLE 2

| | | GGDS | GGDS | GGDS |
|---|---|---|---|---|
| | | median | range | p-value |
| Gender | | | | |
| women | N = 13 | 0.0611 | 0.0045-0.1841 | 0.738* |
| men | N = 31 | 0.0483 | 0.0030-0.2043 | |
| Age | N = 44 | | | |
| median | 68.1 y | 0.0493 | 0.0030-0.2043 | 0.834** |
| range | 25.8-81.2 y | | | |
| Smoking Status | | | | |
| active | N = 14 | 0.0506 | 0.0045-0.1452 | 0.399*** |
| former | N = 24 | 0.0515 | 0.0030-0.2043 | |
| never | N = 6 | 0.0395 | 0.0092-0.0975 | |
| Histopathology | | | | |
| squamous | N = 21 | 0.0462 | 0.0030-0.2043 | 0.290* |
| non-squamous | N = 23 | 0.0527 | 0.0077-0.1870 | |
| pStage | | | | |
| I | N = 24 | 0.0515 | 0.0045-0.2043 | 0.964*** |
| II | N = 11 | 0.0462 | 0.0030-0.1739 | |
| III/IV | N = 9 | 0.0476 | 0.0102-0.1339 | |
| ECOG-PS | | | | |
| 0 | N = 20 | 0.0464 | 0.0030-0.1452 | 0.305* |
| >0 | N = 24 | 0.0577 | 0.0077-0.2043 | |
| Weight Loss | | | | |
| (>5% in 3 | | | | |
| months) | | | | |
| absent | N = 37 | 0.0462 | 0.0030-0.2043 | not |
| present | N = 3 | 0.0727 | 0.0483-0.1452 | done |
| unknown | N = 4 | 0.0725 | 0.0078-0.1739 | |

*Wilcoxon Rank Sum test;
**Spearman correlation coefficient;
***Kuskal Wallis test.

In order to assess whether GGDS would be predictive of OS and DFS of patients with completely resected NSCLC, the cohort was dichotomized into high versus low GGDS based on the cohort median (0.049). OS was shown to be significantly different (p=007, N=44) while DFS was marginally different (p=0.135, N=38).

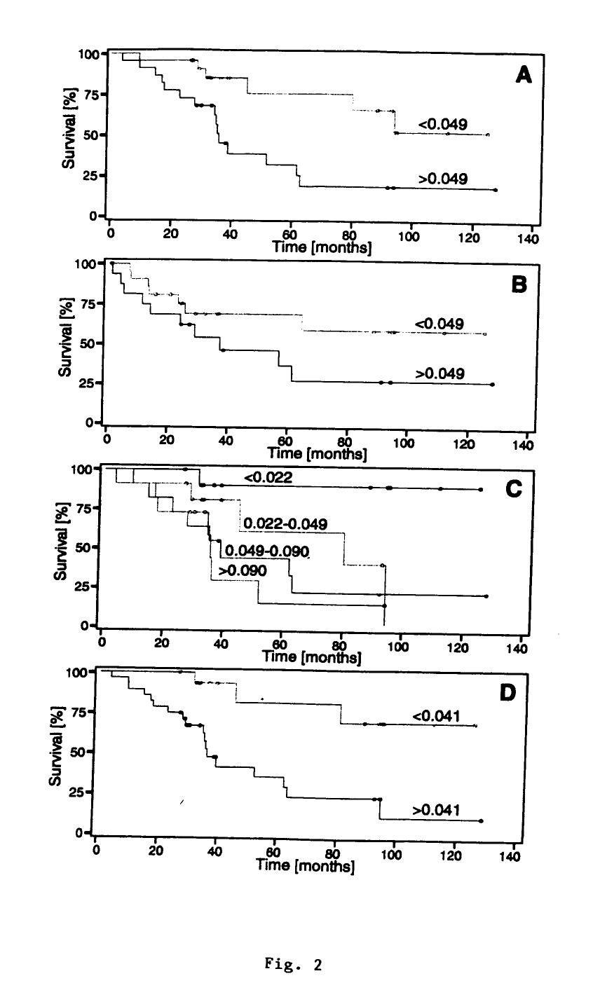

The results of the Kaplan-Meier survival curves shown in FIG. 2A (OS) and FIG. 2B (DFS) demonstrate that patients with low GGDS (<0.049) lived longer and had disease recurrence later than those with high GGDS (>0.049).

The results of the Kaplan-Meier survival curves shown in FIG. 2C (OS) demonstrate that when the cohort was divided into quartiles of 11 patients each, the group with the lowest GGDS (group 1: 0.003-0.0151) had the best OS (p=0.019) compared to the other three quartiles (group 2: 0.0285-0.0483; group 3: 0.0503-0.0889; group 4:0.0911-0.2043). In fact, only one patient in group 1 had died after 31.2 months from recurrent disease compared to 5 patients in group 2, 7 patients in group 3, end 8 patients in group 4.

The results of the Kaplan-Meier survival curves shown in FIG. 2D (OS) demonstrate that when the cohort was divided into quartiles using the optimal cut point of GGDS=0.041, 16 patients had low GGDS (0.003-0.0401) and 28 had high GGDS (0.042-0.2043) with a p-value of 0.0023 for OS. Even after adjusting for multiple cut point analyses the p-value was still 0.031 for OS. In this group of patients, GGDS was not significantly associated with patients' age, gender, cigarette use, tumor stage, tumor histology, or performance status (Table 2).

### Discussion

This study of global genome damage analysis for human epithelial malignancy convincingly demonstrates a statistically significant and clinically meaningful association with the in vivo tumor phenotype. This shows that the clinical behavior of tumors with low GGDS was relatively benign while tumors with high GGDS are aggressive resulting in early death of patients. Since GGDS determination is a robust and reliable technology, it can easily be integrated into clinical decisions on cancer care. For instance, adjuvant treatment of epithelial cancer benefits only a minority of patients while toxicity is substantial. GGDS may prove useful in selecting patients at high risk for tumor associated mortality for adjuvant therapeutic interventions.

### Incorporation by Reference

The invention is not to be limited in scope by the specific embodiments described which are intended as single illustrations of individual aspects of the invention, and functionally equivalent methods and components are within the scope of the invention.

Indeed various modifications of the invention, in addition to those shown and described herein will become apparent to those skilled in the art from the foregoing description and accompanying drawings. Such modifications are intended to fall within the scope of the appended claims.

All references cited herein, including patent applications, patents, and other publications, are incorporated by reference herein in their entireties for all purposes.

1. A method for determining phenotype of a cancer in a subject comprising determining a global genome damage score (hereinafter “GGDS”) for the cancer, wherein said GGDS is a relative measure of (a) number of heterozygous single nucleotide polymorphisms (“SNPs”) in a plurality of heterozygous SNPs, said plurality of heterozygous SNPs consisting of different SNPs wherein heterozygosity occurs in genomic DNA of non-cancerous tissue of said species to which said subject belongs, wherein said number of heterozygous SNPs in said plurality is in excess of 100 SNPs; and (b) the number of SNPs for which heterozygosity is determined to be present, or the number of SNPs for which heterozygosity is determined to be absent, among the number of heterozygous SNPs in said plurality of (a), in a nucleic acid sample of, or derived from, genomic DNA of cancerous tissue of the subject.

2. The method of claim 1 wherein said number of SNPs in (b), for which heterozygosity is determined to be present or for which heterozygosity is determined to be absent, is determined by a second method comprising
a) contacting under hybridization conditions said nucleic acid sample of, or derived from, genomic DNA of cancerous tissue of the subject independently with each member of a SNP pair, for each heterozygous SNP in said plurality of heterozygous SNPs, each SNP pair being a pair of oligonucleotides differing in sequence at a single nucleotide position that is a site of a single nucleotide polymorphism; and
b) detecting any hybridization that occurs.

3. The method of claim 1 wherein the plurality of heterozygous SNPs comprises SNPs comprising a nucleotide sequence complementary to the genomic DNA sequence of at least 100 different loci in said species.

4. The method of claim 1 wherein the plurality of heterozygous SNPs comprises at least 100 SNPs that are randomly distributed throughout the genome at least every 500 kb pairs.

5. The method of claim 1 wherein the plurality of heterozygous SNPs comprises at least 100 SNPs that are not within the same 500 kb region of said genomic DNA as any other SNPs within said plurality.

6. The method of claim 1 wherein the plurality of heterozygous SNPs is not found in regions of genomic DNA that are repetitive.

7. The method of claim 1 wherein the plurality of heterozygous SNPs comprises at least one SNP on each of the 23 human chromosome pairs.

8. The method of claim 1 wherein the plurality of heterozygous SNPs comprises at least one SNP on each arm of each of the 23 human chromosome pairs.

9. The method of claim 1 wherein the plurality of heterozygous SNPs comprises SNPs located in the genome on different chromosomal loci, respectively, and wherein the different chromosomal loci comprise loci on each of the chromosomes of said species.

10. The method of claim 1 wherein said non-cancerous tissue is the same tissue type as said cancerous tissue.

11. The method of claim 1 wherein said non-cancerous tissue is not the same tissue type as said cancerous tissue.

12. The method of claim 1 wherein said non-cancerous tissue is mononuclear blood cells or saliva cells.

13. The method of claim 1 wherein said non-cancerous tissue is from the subject.

14. The method of claim 1 wherein the non-cancerous tissue is from a plurality of different organisms.

15. The method of claim 1 wherein the subject is human.

16. The method of claim 1 wherein said number of SNPs in (b), for which heterozygosity is determined to be present or for which heterozygosity is determined to be absent, is determined by a method that does not comprise detecting a change in size of restriction enzyme-digested nucleic acid fragments.

17. The method of claim 1 wherein said relative measure is the number of said SNPs in (b) for which heterozygosity is determined to be absent divided by the number of heterozygous SNPs in said plurality in (a).

18. The method of claim 1 wherein the cancer is an epithelial cancer.

19. The method of claim 18 wherein the epithelial cancer is breast cancer, prostate cancer, lung cancer, or colon cancer.

20. The method of claim 18 wherein the epithelial cancer is non-small cell lung carcinoma.

21. The method of claim 1 wherein the phenotype is predicted response to therapy.

22. The method of claim 21 wherein the therapy is chemotherapy or radiation therapy

23. The method of claim 21 wherein the therapy is immunotherapy.

24. The method of claim 1 wherein the phenotype is predicted probability of survival.

25. The method of claim 1 wherein the phenotype is predicted probability of metastasis within a given time period.

26. The method of claim 1 wherein the phenotype is predicted probability of tumor recurrence.

27. The method of claim 2 wherein said second method comprises prior to said contacting step the step of producing said nucleic acid sample by a third method comprising amplifying genomic DNA of cancerous tissue of the subject.

28. The method of claim 1 or 9 wherein said number of heterozygous SNPs in said plurality is in excess of 500.

29. The method of claim 1 or 9 wherein said number of heterozygous SNPs in said plurality is in excess of 1000.

30. The method of claim 1 wherein the plurality of heterozygous SNPs comprises at least 500 SNPs that are not within the same 500 kb region of said genomic DNA as any other SNPs within said plurality.

31. A kit comprising:
a) nucleic acid probes comprising SNP hybridization probes, said SNP hybridization probes comprising nucleotide sequences complementary to a plurality of SNPs, respectively, said SNPs consisting of at least 100 different SNPs wherein heterozygosity occurs in genomic DNA of non-cancerous tissue of the same species; and
b) a computer program product for use in conjunction with a computer system, the computer program product comprising a computer readable storage medium and a computer program mechanism embedded therein, the computer program mechanism comprising instructions for determining a relative measure of (i) the number of at least 100 different SNPs in (a), and (ii) the number of SNPs for which heterozygosity is determined to be present, or the number of SNPs for which heterozygosity is determined to be absent, among the at least 100 different SNPs of (a) in a nucleic acid sample of, or derived from, genomic DNA of cancerous tissue of a subject of said species.

32. The kit of claim 31 which comprises said nucleic acid probes attached to a solid or semi-solid phase.

33. A method for determining the probability of progression to cancer of pre-cancerous tissue in a subject comprising determining a GGDS for the precancerous tissue, wherein said GGDS is a relative measure of (a) number of heterozygous SNPs in a plurality of heterozygous SNPs, said plurality of heterozygous SNPs consisting of different SNPs wherein heterozygosity occurs in genomic DNA of non-cancerous tissue of said species to which said subject belongs, wherein said number of heterozygous SNPs in said plurality is in excess of 100 SNPs; and (b) the number of SNPs for which heterozygosity is determined to be present, or the number of SNPs for which heterozygosity is determined to be absent, among the number of heterozygous SNPs in said plurality of (a), in a nucleic acid sample of, or derived from, genomic DNA of precancerous tissue of the subject.

34. A computer comprising:
a central processing unit;
a memory, coupled to the central processing unit, the memory storing:
(i) instructions for computing a GGDS for cancerous or precancerous tissue, wherein said GGDS is a relative measure of (a) number of heterozygous SNPs in a plurality of heterozygous SNPs, said plurality of heterozygous SNPs consisting of different SNPs wherein heterozygosity occurs in genomic DNA of non-cancerous tissue of said species to which said subject belongs, wherein said number of heterozygous SNPs in said plurality is in excess of 100 SNPs; and (b) the number of SNPs for which heterozygosity is determined to be present, or the number of SNPs for which heterozygosity is determined to be absent, among the number of heterozygous SNPs in said plurality of (a), in a nucleic acid sample of, or derived from, genomic DNA of cancerous or precancerous tissue of the subject.

35. The computer of claim 34, the memory further storing:
(ii) instructions for comparing said GGDS to a threshold value; and
(iii) instructions for outputing an indication of whether said GGDS is above or below a threshold value, or a phenotype based on said indication.

36. The computer of claim 34, the memory further storing in a database said number of heterozygous SNPs of (a).

37. The computer of claim 36, wherein the memory further stores in a database an indication of the identity of each SNP in the heterozygous SNPs of (a).

38. The computer of claim 37, wherein the number of heterozygous SNPs of (a) comprises heterozygous SNPs from noncancerous tissue of a plurality of members of said species, and wherein said identity of each heterozygous SNP in the database is associated with an identifier for which organism exhibits said heterozygous SNP.

39. The computer of claim 34 or 35, wherein said memory further stores:
(i) instructions for receiving SNP probe hybridization data;
(ii) instructions for storing SNP probe hybridization data;
(iii) instructions for comparing SNP probe hybridization data to determine whether an absence or presence of SNP heterozygosity has occurred in said nucleic acid sample from cancerous or precancerous tissue.

40. A computer program product for use in conjunction with a computer system, the computer program product comprising a computer readable storage medium and a computer program mechanism embedded therein, the computer program mechanism comprising:
(i) instructions for computing a GGDS for cancerous or precancerous tissue, wherein said GGDS is a relative measure of (a) number of heterozygous SNPs in a plurality of heterozygous SNPs, said plurality of heterozygous SNPs consisting of different SNPs wherein heterozygosity occurs in genomic DNA of non-cancerous tissue of said species to which said subject belongs, wherein said number of heterozygous SNPs in said plurality is in excess of 100 SNPs; and (b) the number of SNPs for which heterozygosity is determined to be present, or the number of SNPs for which heterozygosity is determined to be absent, among the number of heterozygous SNPs in said plurality of (a), in a nucleic acid sample of, or derived from, genomic DNA of cancerous or precancerous tissue of the subject.

41. The computer program product of claim 40, wherein the computer program mechanism further comprises:
(ii) instructions for comparing said GGDS to a threshold value; and
(iii) instructions for outputing an indication of whether said GGDS is above or below a threshold value, or a phenotype based on said indication.

42. The computer program product of claim 40, the memory further storing in a database said number of heterozygous SNPs of (a).

43. The computer program product of claim 42, wherein the memory further stores in a database an indication of the identity of each SNP in the heterozygous SNPs of (a).

44. The computer program product of claim 43, wherein the number of heterozygous SNPs of (a) comprises heterozygous SNPs from noncancerous tissue of a plurality of members of said species, and wherein said identity of each heterozygous SNP in the database is associated with an identifier for which organism exhibits said heterozygous SNP.

45. The computer program product of claim 40 or 41, wherein said memory further stores:
(i) instructions for receiving SNP probe hybridization data;
(ii) instructions for storing SNP probe hybridization data;
(iii) instructions for comparing SNP probe hybridization data to determine whether an absence or presence of SNP heterozygosity has occurred in said nucleic acid sample from cancerous or precancerous tissue.
